(19) United States
(12) Patent Application Publication
(10) Pub. No.: US 2025/0134819 A1
(43) Pub. Date: May 1, 2025

(54) ORAL DOSAGE FORM WITH A GASTRO RESISTANT CAPSULE SHELL

(71) Applicant: SOCIETE DES PRODUITS NESTLE S.A., Vevey (CH)

(72) Inventors: GERARD VINYES PARES, Alella (ES); STEFANIE RAU, Olching (DE)

(21) Appl. No.: 18/835,527

(22) PCT Filed: Feb. 1, 2023
(86) PCT No.: PCT/EP2023/052413

§ 371 Date: Aug. 2, 2024

(30) Foreign Application Priority Data
Feb. 3, 2022 (EP) 22154908.2

(51) Int. Cl. A61K 9/48; A61K 38/48

(52) U.S. Cl. CPC A61K 9/4825; A61K 9/4816; A61K 38/482; A61K 38/4826; A61K 38/4873; A61K 38/4886

(57) ABSTRACT

The present invention relates to a gastro resistant capsule comprising one or more proteases. wherein the capsule shell comprises a water-soluble film forming polymer and a water-soluble enteric polymer. The present invention also relates to methods of manufacturing the gastro resistant capsules and uses thereof.

### FIELD OF THE INVENTION

The present invention relates to gastro resistant capsules comprising one or more proteases. The present invention also relates to methods of manufacturing the gastro resistant capsules and uses thereof.

### BACKGROUND TO THE INVENTION

Oral administration of proteolytic enzymes has been shown to be associated with beneficial immunomodulatory effects. For example, in a randomized trial of subjects with moderate-to-severe knee osteoarthritis, oral administration of proteolytic enzymes were found to have comparable effectiveness to diclofenac in relieving pain and increasing function (Ueberall, M.A., et al., 2016. Journal of pain research, 9, pp. 941-961).

Proteolytic enzymes have also been shown as a highly efficient strengthening factor for antibiotic therapy of urogenital chlamydiosis (Sukhikh, G. T., et al., 1997. International journal of immunotherapy, 13(3-4), pp. 131-133); to provide an additional beneficial effect for the patients suffering from cervical syndrome (Tilscher, H., et al., 1996. Wiener medizinische Wochenschrift, 146(5), pp. 91-95); and to decrease incidence of obstructive bronchitis in children with recurrent obstructive bronchitis (Lanchava, N., et al., 2005. Georgian medical news, 127, pp. 50-53).

Presently, proteolytic enzymes are usually administered orally in the form of enterically coated tablets. However, in order to receive an effective dosage this can require subjects to receive six tablets or more daily (e.g. two tablets, three times daily) (Ueberall, M.A., et al., 2016. Journal of pain research, 9, pp. 941-961). It can be challenging for subjects to adhere to such a dosage schedule and it would desirable to provide an oral dosage form which can be administered fewer times whilst providing an effective dosage.

### SUMMARY OF THE INVENTION

The present inventors have developed an oral dosage form which requires fewer administrations to deliver an effective dosage of proteolytic enzymes.

The present inventors have surprisingly found that a gastro resistant capsule shell comprising a water-soluble film forming polymer (e.g. HPMC) and a water-soluble enteric polymer (e.g. pectin) releases the proteolytic enzymes in the upper side of the small intestine and mimics the kinetic profile of an enterically coated tablet. In contrast, a gastro resistant capsule shell comprising a water-soluble film forming polymer (e.g. HPMC) and gellan gum did not release the proteolytic enzymes at the right point in the gastrointestinal tract.

In one aspect, the present invention provides a capsule comprising one or more proteases, wherein the capsule shell comprises a water-soluble film forming polymer and a water-soluble enteric polymer. Preferably, the capsule is acid resistant. More preferably, the capsule is gastro resistant.

In another aspect, the present invention provides a method of manufacturing a capsule comprising one or more proteases, the method comprising:
(a) providing a capsule shell comprising a water-soluble film forming polymer and a water-soluble enteric polymer, preferably wherein the capsule shell is acid resistant, more preferably wherein the capsule shell is gastro resistant; and
(b) filling the capsule shell with a capsule composition comprising one or more proteases.

In another aspect, the present invention provides a capsule obtained or obtainable by a method of manufacture of the present invention.

The capsule shell may be insoluble under gastric conditions and soluble under small intestinal conditions. Suitably, gastric conditions are about pH 2.0 at about 37° C. and/or small intestinal conditions are from about pH 5.5 to about pH 7.0 at about 37° C.

The capsule shell may comprise the water-soluble film forming polymer in an amount of from about 65 wt % to about 97 wt %; the water-soluble enteric polymer in an amount of from about 3 wt % to about 30 wt %; and/or have a moisture content of from about 4 wt % to about 7 wt %.

Any suitable water-soluble enteric polymer may be used. The water-soluble enteric polymer may be selected from one or more of pectin, propylene glycol alginate (PGA), or xanthum gum. In some embodiments, the water-soluble enteric polymer is pectin. Suitably, the pectin has a degree of esterification of from about 15% to about 40%, from about 20% to about 35%, or from about 20% to about 30%. Suitably, the pectin has a degree of amidation of from about 0% to about 25%, from about 10% to about 25%, or from about 15% to about 25%. Suitably, the water-soluble enteric polymer has a molecular weight of from about 20 kDa to about 1000 kDa, from about 40 kDa to about 400 kDa, or from about 50 kDa to about 200 kDa.

Any suitable water-soluble film forming polymer may be used. The water-soluble film forming polymer may be selected from one or more of hydroxypropyl methylcellulose (HPMC), starch, gelatin, pullulan, polyvinyl alcohol, hydroxypropylated starch, hydroxyethylated starch, hydroxypropyl cellulose, methylcellulose, hydroxyethyl cellulose, and hydroxyethyl methyl cellulose. In some embodiments, the water-soluble film forming polymer is hydroxypropyl methylcellulose (HPMC). Suitably, the water-soluble film forming polymer has a molecular weight of from about 50 kDa to about 815 kDa, or from about 50 kDa to about 400 kDa.

In some embodiments, the capsule shell further comprises a coagulant, such as gellan gum, carrageen, agar, konjac gum, or locust beam gum, optionally in an amount of from about 0.5 wt % to about 3 wt %.

In some embodiments, the capsule shell does not comprise a coagulant. In some embodiments, the capsule shell does not comprise gellan gum and/or carrageenan.

Any suitable proteases may be used. The one or more proteases may be selected from the group consisting of: bromelain, trypsin, papain, pancreatin, chymotrypsin, serratiopeptidase, ficain, nattokinase, or any combination thereof. In some embodiments, the one or more proteases comprise or consist of bromelain and/or trypsin. In some embodiments, the one or more proteases comprise or consist of bromelain and trypsin.

The one or more proteases may be present in any suitable amount. Suitably, the capsule comprises the one or more proteases in a total amount of from about 50 mg to about 1000 mg, from about 75 mg to about 800 mg, from about 100 mg to about 600 mg, from about 125 mg to about 500 mg, from about 150 mg to about 400 mg, or from about 200 mg to about 300 mg. Suitably, the capsule comprises the one or more proteases in a total amount of from about 1000 FIP units to about 12000 FIP units, from about 1500 FIP units to about 10000 FIP units, from about 2000 FIP units to about 8000 FIP units, from about 2500 FIP units to about 6000 FIP units, or from about 3000 FIP units to about 4000 FIP units. Suitably, the capsule comprises bromelain in an amount of from about 50 mg to about 500 mg, from about 75 mg to about 400 mg, from about 100 mg to about 300 mg, or from about 125 mg to about 200 mg. Suitably, the capsule comprises bromelain in an amount of from about 400 FIP units to about 3000 FIP units, from about 500 FIP units to about 2500 FIP units, from about 600 FIP units to about 2000 FIP units, from about 700 FIP units to about 1500 FIP units, or from about 800 FIP units to about 1000 FIP units. Suitably, the capsule comprises trypsin in an amount of from about 20 mg to about 250 mg, from about 30 mg to about 200 mg, from about 40 mg to about 150 mg, from about 50 mg to about 100 mg, or from about 60 mg to about 80 mg. Suitably, the capsule comprises trypsin in an amount of from about 1000 FIP units to about 10000 FIP units, from about 1500 FIP units to about 8000 FIP units, from about 1750 FIP units to about 6000 FIP units, from about 2000 FIP units to about 5000 FIP units, or from about 2500 FIP units to about 4000 FIP units.

In some embodiments, the capsule comprises bromelain in an amount of about 150 mg and/or trypsin in an amount of about 72 mg. In some embodiments, the capsule comprises bromelain in an amount of about 900 FIP units and/or trypsin in an amount of about 2880 FIP units.

The capsule may comprise one or more flavonoid. Any suitable flavonoid may be used in any suitable amount. Suitably, the one or more flavonoid is selected from rutin, quercetin, kaempferol, myricetin, fisetin, catechin, epicatechin, hydroxytyrosol, oleuropein, hesperidin, or any combination thereof. In some embodiments, the one or more flavonoid comprises or consists of rutin. Suitably, the capsule comprises the one or more flavonoid in a total amount of from about 50 mg to about 1000 mg, from about 100 mg to about 600 mg, from about 125 mg to about 400 mg, or from about 150 mg to about 300 mg. In some embodiments, the capsule comprises the one or more flavonoid in a total amount of about 200 mg.

The capsule may comprise one or more bulking agent. Any suitable bulking agent may be used in any suitable amount. Suitably, the one or more bulking agent comprises or consists of one or more cellulose derivative, such as powdered and microcrystalline cellulose (MCC), ethyl cellulose, hydroxypropyl cellulose (HPC), hydroxyethyl cellulose (HEC), hydroxypropyl methylcellulose (HPMC), HPMC phthalate, HPMC acetate succinate, cellulose acetate (CA), CA phthalate, sodium (Na) and calcium (Ca) carboxymethylcellulose (CMC), croscarmellose sodium (XCMCNa), methyl cellulose, and low substituted HPC, or any combination thereof. In some embodiments, the one or more bulking agent comprises or consists of microcrystalline cellulose (MCC) and/or hydroxypropyl cellulose (HPC).

The capsule may comprise one or more further component. Suitably, the capsule further comprises one or more antioxidant, such as L-ascorbyl palmitate. Suitably, the capsule further comprises medium chain triglycerides.

The capsule may remain substantially intact in the gastric phase and/or the capsule may be substantially dissolved in the small intestinal phase. Suitably, the capsule remains fully intact in the gastric phase and/or the capsule is fully dissolved in the small intestinal phase. Suitably, the protease activity is not substantially released in the gastric phase and/or the protease activity is not substantially released in the small intestinal phase. Suitably, the protease activity is not released in the gastric phase and/or the protease activity is fully released in the small intestinal phase. Suitably, the protease activity is not released in the gastric phase and the protease activity is fully released in the small intestinal phase.

In another aspect, the present invention provides a pack comprising a plurality of capsules of the present invention. Suitably, the pack comprises about 45 or more capsules, about 90 or more capsules, about 135 or more capsules, about 180 or more capsules, or about 270 or more capsules. Suitably, the pack comprises about 15 daily doses or more, about 30 daily doses or more, about 45 daily doses or more, about 60 daily doses or more, or about 90 daily doses or more.

In another aspect, the present invention provides use of a capsule of the present invention as a nutritional supplement.

In another aspect, the present invention provides a capsule of the present invention for use as a medicament.

In another aspect, the present invention provides a capsule of the present invention for use in treating and/or preventing an inflammatory disease, joint pain, arthrosis, injuries, back pain, or sciatica, or for use in accelerating healing.

In another aspect, the present invention provides a of the present invention for use in treating and/or preventing an inflammatory disease, such as phlebitis, prostate inflammation, or cystitis.

In another aspect, the present invention provides a capsule of the present invention for use in treating and/or preventing joint pain, such as joint pain in the shoulder, joint pain in the elbow, joint pain in the fingers, joint pain in the knee, menopausal joint pain, or rheumatic pain.

In another aspect, the present invention provides a capsule of the present invention for use in treating and/or preventing arthrosis, arthritis, or osteoarthritis, such as osteoarthritis in the hip, osteoarthritis in the knee, arthrosis in the metatarsophalangeal joint of the big toe, osteoarthritis in the finger, osteoarthritis in the foot, osteoarthritis of the ankle, or Heberden and Bouchard nodes.

In another aspect, the present invention provides a capsule of the present invention for use in treating and/or preventing injuries, such as sports injuries, muscle soreness, strains, overload damage, sprains, or bruises.

In another aspect, the present invention provides a capsule of the present invention for use in treating and/or preventing back pain, such as upper back pain, disc prolapse, or lumbago, or sciatica.

In another aspect, the present invention provides a capsule of the present invention for use in accelerating healing, such as wound healing or healing after operation.

The one or more proteases may be administered in any suitable dose. Suitably, the one or more proteases are administered in a total daily dose of from about 100 mg to about 2000 mg, from about 200 mg to about 1500 mg, from about 300 mg to about 1200 mg, from about 400 mg to about 1000 mg, from about 500 mg to about 900 mg, or from about 600 mg to about 800 mg. Suitably, the one or more proteases are administered in a total daily dose of from about 2000 FIP units to about 30000 FIP units, from about 4000 FIP units to about 25000 FIP units, from about 6000 FIP units to about 20000 FIP units, from about 8000 FIP units to about 15000 FIP units, or from about 10000 FIP units to about 12000 FIP units. Suitably, bromelain is administered in a daily dose of from about 100 mg to about 1000 mg, from about 200 mg to about 900 mg, from about 300 mg to about 800 mg, from about 350 mg to about 700 mg, from about 400 mg to about 600 mg, or from about 450 mg to about 500 mg. Suitably, bromelain is administered in a daily dose of from about 500 FIP units to about 10000 FIP units, from about 1000 FIP units to about 8000 FIP units, from about 1500 FIP units to about 6000 FIP units, from about 2000 FIP units to about 4000 FIP units, or from about 2500 FIP units to about 3000 FIP units. Suitably, trypsin is administered in a daily dose of from about 50 mg to about 500 mg, from about 100 mg to about 400 mg, from about 150 mg to about 350 mg, from about 175 mg to about 300 mg, or from about 200 mg to about 250 mg. Suitably, trypsin is administered in a daily dose of from about 2000 FIP units to about 20000 FIP units, from about 3000 FIP units to about 15000 FIP units, from about 4000 FIP units to about 12000 FIP units, from about 5000 FIP units to about 11000 FIP units, from about 6000 FIP units to about 10000 FIP units, or from about 7000 FIP units to about 9000 FIP units.

In some embodiments, the one or more proteases are administered in a total daily dose of about 666 mg and/or about 11340 FIP units. In some embodiments, bromelain is administered in a daily dose of about 450 mg and/or trypsin is administered in a daily dose of about 216 mg. In some embodiments, bromelain is administered in a daily dose of about 2700 FIP units and/or trypsin is administered in a daily dose of about 8640 FIP units.

Suitably, rutin is administered in a total daily dose of from about 100 mg to about 1200 mg, from about 200 mg to about 1000 mg, from about 300 mg to about 900 mg, from about 400 mg to about 800 mg, from about 500 mg to about 700 mg. In some embodiments, rutin is administered in a total daily dose of about 600 mg.

Suitably, the capsule is formulated to comprise about ½ a daily dose, about ⅓ of a daily dose, about ¼ of a daily dose, or about ⅕ of a daily dose. In some embodiments, the capsule is formulated to comprise about ½ a daily dose, or about ⅓ of a daily dose. In some embodiments, the capsule is formulated to comprise about ⅓ of a daily dose.

Suitably, five or fewer, four or fewer, or three or fewer capsules are administered daily. In some embodiments, two or three capsules are administered daily. In some embodiments, three capsules are administered daily. The capsules may be administered simultaneously or separately, or any combination thereof.

Suitably, the capsule is administered in a fasting state, optionally at least about 45 minutes before a meal. Suitably, the capsule is administered for at least 15 days.

In another aspect, the present invention provides use of a capsule shell comprising a water-soluble film forming polymer and a water-soluble enteric polymer to deliver one or more proteases. The one or more proteases may be delivered to the small intestine. The capsule shell and one or more proteases may be any described herein.

In another aspect, the present invention provides use of a capsule to deliver one or more proteases, wherein the capsule shell comprises a water-soluble film forming polymer and a water-soluble enteric polymer. The one or more proteases may be delivered to the small intestine. The capsule shell and one or more proteases may be any described herein.

### DESCRIPTION OF DRAWINGS

FIG. 1—Activity of the Wobenzym PS tablet along the entire upper GIT

Trypsin (A-B; USP units/reactor) and bromelain (C-D; units/reactor) activity of the Wobenzym PS tablet along the entire upper gastrointestinal tract (GIT). Average (A and C, presented as mean±stdev, n=3) and single replicate (B and D) measurements have been presented. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

FIG. 2—Activity of the Wobenzym control HPMC capsule along the entire upper GIT

Trypsin (A-B; USP units/reactor) and bromelain (C-D, units per reactor) activity of the Wobenzym control HPMC capsule along the entire upper GIT. Average (A and C, presented as mean±stdev, n=3) and single replicate (B and D) measurements have been presented. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

FIG. 3—Activity of the Wobenzym HPMC/gellan gum capsule along the entire upper GIT

Trypsin (A-B; USP units/reactor) and bromelain (C-D; units/reactor) activity of the Wobenzym HPMC/gellan gum capsule along the entire upper GIT. Average (A and C, presented as mean±stdev, n=3) and single replicate (B and D) measurements have been presented. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

FIG. 4—Activity of the Wobenzym HPMC/pectin capsule along the entire upper GIT

Trypsin (A-B; USP units/reactor) and bromelain (C-D; units/reactor) activity of the Wobenzym HPMC/pectin capsule along the entire upper GIT. Average (A and C, presented as mean±stdev, n=3) and single replicate (B and D) measurements have been presented. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

FIG. 5—Dissolution behaviour of the Wobenzym HPMC/pectin capsule

Pictures are included for a few distinct time points (i.e., product as such, ST45, SI60, SI120, and SI180) and the corresponding capsule scores are included in the top left corner of each picture.

FIG. 6—Daily trypsin activity for each product formulation

Average daily trypsin activity (USP units/day, presented as mean±stdev, n=3) of the different product formulations along the entire upper GIT. The results have been presented for (A) Wobenzym PS tablet, (B) Wobenzym control HPMC capsule, (C) Wobenzym HPMC/gellan gum capsule, and (D) Wobenzym HPMC/pectin capsule. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

FIG. 7—Daily bromelain activity for each product formulation

Average daily bromelain activity (units/day, presented as mean±stdev, n=3) of the different product formulations along the entire upper GIT. The results have been presented for (A) Wobenzym PS tablet, (B) Wobenzym control HPMC capsule, (C) Wobenzym HPMC/gellan gum capsule, and (D) Wobenzym HPMC/pectin capsule. Data are representative for the product as such and the samples collected during the gastric phase (ST 15 min, ST 30 min, ST 45 min) and the small intestinal phase (SI 30 min, SI 60 min, SI 90 min, SI 120 min, SI 150 min, and SI 180 min). Furthermore, the capsule integrity scores at each sampling point are indicated above the bars in the figures (1=intact to 4=fully dissolved).

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

As used herein, the “FIP unit” refers to the protease activity units as defined by the Fédération Internationale Pharmaceutique (International Union of Biochemistry. Nomenclature Committee: Enzyme Nomenclature. London: Academic Press; 1984).

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

All publications mentioned in the specification are herein incorporated by reference.

### Capsule

In one aspect, the present invention provides a capsule comprising one or more proteases.

As used herein, a “capsule” may refer to a dosage form in which capsule contents (comprising one or more active agents) are encapsulated in a capsule shell. A “capsule shell” may be formed of a body portion and a cap portion. The capsule of the present invention may be a hard-shelled capsule. A hard capsule shell is formed without capsule contents and subsequently filled with capsule contents (comprising one or more active agents). Following administration, the hard capsule shell dissolves, releasing the capsule contents (comprising one or more active agents) encapsulated therein.

The capsule may be acid resistant. As used herein, an “acid resistant capsule” may refer to a capsule in which the capsule contents (e.g. the one or more active agents) are not released in an acidic environment. The shell of an acid resistant capsule may be insoluble in an acidic environment.

The capsule may be gastro resistant. As used herein, a “gastro resistant capsule” may refer to a capsule in which the capsule contents (e.g. the one or more active agents) are not released in the stomach but are released further down the gastrointestinal tract. The shell of a gastro resistant capsule may be insoluble in gastric acid. Gastric acid (also known as gastric juice or stomach acid) is a digestive fluid formed within the stomach lining and typically has a pH of from about 1 to about 3.

Suitably, the capsule shell used in the present invention is insoluble under gastric conditions. As used herein, “gastric conditions” may refer to the highly acid environment in the stomach lumen (Evans, D. F., et al., 1988. Gut, 29(8), pp. 1035-1041). Suitably, gastric conditions may be from about pH 1 to about pH 3 (e.g. about pH 2.0) at about 37° C.

Suitably, the capsule shell used in the present invention is soluble under small intestinal conditions. As used herein, “small intestinal conditions” may refer to the environment in the small intestine (including the duodenum, jejunum, and ileum). The pH gradually increases in the small intestine from about pH 6 to about pH 7.4 in the terminal ileum (Fallingborg, J., 1999.Danish medical bulletin, 46(3), pp. 183-196). Suitably, small intestinal conditions may be from about pH 5 to about pH 8 (e.g. from about pH 5.5 to about pH 7.0) at about 37° C.

In some embodiments, the capsule used in the present invention is an HPMC capsule. As used herein, an “HPMC capsule” may refer to a capsule in which the capsule shell comprises hydroxypropyl methylcellulose (HPMC). See e.g. Majee, S. B., et al., 2017. Int J Pharm Pharm Sci, 9(10), pp. 1-6.

Suitably, the capsule used in the present invention is not an enteric coated capsule. As used herein, an “enteric coated capsule” may refer to a capsule with an enteric coating (see e.g. Cole, E. T., et al., 2002. International journal of pharmaceutics, 231(1), pp. 83-95). An “enteric coating” may refer to a polymer barrier applied to oral medication that prevents its dissolution or disintegration in the gastric environment. Materials used for enteric coatings include fatty acids, waxes, shellac, plastics, and plant fibers. Polymers commonly used to as enteric coating include anionic polymethacrylates, cellulose based polymers (e.g. cellulose acetate phthalate), and polyvinyl derivatives (e.g. polyvinyl acetate phthalate).

### Capsule Shell

### Water-Soluble Film Forming Polymer

The capsule shell used in the present invention comprises a water-soluble film forming polymer.

As used herein, “a water-soluble film forming polymer” may refer to a water-soluble polymer which can be used to form film. Exemplary water-soluble film forming polymers include hydroxypropyl methylcellulose, starch, gelatin, pullulan, polyvinyl alcohol, hydroxypropylated starch, hydroxyethylated starch, hydroxypropyl cellulose, methylcellulose, hydroxyethyl cellulose, and hydroxyethyl methyl cellulose.

Suitably, the water-soluble film forming polymer may have a molecular weight of at least about 50 kDa. Suitably, the water-soluble film forming polymer may have a molecular weight of from about 50 kDa to about 815 kDa, or from about 50 kDa to about 400 kDa.

In some embodiments, the capsule shell used in the present invention comprises hydroxypropyl methylcellulose (HPMC).

Hydroxypropyl methylcellulose (also known as hypromellose) is a methyl and hydroxypropyl mixed ether of cellulose which has been used widely as a capsule shell material (see e.g. Majee, S. B., et al., 2017. Int J Pharm Pharm Sci, 9(10), pp. 1-6).

Suitably, the HPMC (or other water-soluble film forming polymer) is present in the capsule shell in an amount of at least about 65 wt %, at least about 70 wt %, at least about 75 wt %, or at least about 80 wt %.

Suitably, the HPMC (or other water-soluble film forming polymer) is present in the capsule shell in an amount of about 97 wt % or less, about 95 wt % or less, about 90 wt % or less, or about 85 wt % or less.

Suitably, the HPMC (or other water-soluble film forming polymer) is present in the capsule shell in an amount of from about 65 wt % to about 97 wt %, from about 70 wt % to about 95 wt %, from about 75 wt % to about 90 wt %, or from about 80 wt % to about 85 wt %.

### Water-Soluble Enteric Polymer

The capsule shell used in the present invention comprises a water-soluble enteric polymer.

As used herein, “a water-soluble enteric polymer” may refer to a water-soluble polymer which is resistant to gastric acid. Suitably, a water-soluble enteric polymer is insoluble in gastric acid. A water-soluble enteric polymer may provide the capsule shell with gastric resistance. Exemplary water-soluble enteric polymers include pectin, propylene glycol alginate (PGA), and xanthan gum. Suitably, the water-soluble enteric polymer is pectin or PGA.

Suitably, the water-soluble enteric polymer has a molecular weight of at least about 20 kDa, at least about 30 kDa, at least about 40 kDa, or at least about 50 kDa. Suitably, the water-soluble enteric polymer has a molecular weight of about 1000 kDa or less, about 800 kDa or less, about 600 kDa or less, about 400 kDa or less, or about 200 kDa or less. Suitably, the water-soluble enteric polymer has a molecular weight of from about 20 kDa to about 1000kDa, from about 40 kDa to about 400 kDa, or from about 50 kDa to about 200 kDa.

Suitably, the water-soluble enteric polymer has a ratio of hydrophobic functional groups to hydrophilic functional groups of from about 30:70 to about 70:30, from about 40:60 to about 60:40, or from about 45:55 to about 55:45. The ratio of hydrophobic functional groups to hydrophilic functional groups may be determined by any suitable method.

In some embodiments, the capsule shell used in the present invention comprises pectin.

Pectins, also known as pectic polysaccharides, are rich in galacturonic acid. Pectins can include homogalacturonan, rhamnogalacturonan-I, and rhamnogalacturonan-II structural elements. Homogalacturonan is the most abundant pectin and is a homopolymer of up to 200 units of α(1-4)-linked D-galacturonic acid. Rhamnogalacturonan-I pectins contain a backbone of the repeating disaccharide: 4)-α-D-galacturonic acid-(1,2)-α-L-rhamnose-(1. Rhamnogalacturonan-II is a less frequent, complex, highly branched polysaccharide.

Suitably, the pectin has a degree of esterification of at least about 15% or at least about 20%. Suitably, the pectin has a degree of esterification of about 40% or less, about 35% or less, or about 30% or less. Suitably, the pectin has a degree of esterification of from about 15% to about 40%, or from about 20% to about 35%, or from about 20% to about 30%.

Suitably, the pectin has a degree of amidation of at least about 0%, at least about 5%, at least about 10%, or at least about 15%. Suitably, the pectin has a degree of amidation of from about 0% to about 25%, from about 5% to about 25%, from about 10% to about 25%, or from about 15% to about 25%.

Suitably, the pectin has a degree of esterification of from about 15% to about 40% and a degree of amidation of from about 0% to about 25%. Suitably, the pectin has a degree of esterification of from about 20% to about 35% and a degree of amidation of from about 10% to about 25%. Suitably, the pectin has a degree of esterification of from about 20% to about 30% and a degree of amidation of from about 15% to about 25%.

The “degree of esterification” (DE) may refer to the percentage of galacturonic acid which is esterified. The degree of esterification can be determined by any method known to the skilled person (see e.g. Guillotin, S.E., et al., 2007. Food hydrocolloids, 21(3), pp. 444-451). For example, the degree of methyl esterification can be determined using the following formula:

Degree of esterification=(millimoles methanol/millimoles uronic acid)×100

where the content of uronic acid is determined using a colorimetric procedure and the methanol content is determined by HPLC following alkali treatment of the pectin sample. For example, as described in Melton, L. D. and Smith, B. G., 2001. Current Protocols in Food Analytical Chemistry, (1), E3-3.

The “degree of amidation” (DA) may refer to the percentage of galacturonic acid which is amidated. The degree of amidation can be determined by any method known to the skilled person. For example, as described in Reitsma, J. C. E., et al., 1986. Food Hydrocolloids, 1(2), pp. 121-127.

Suitably, the pectin (or other water-soluble enteric polymer) is present in the capsule shell in an amount of at least about 3 wt %, at least about 5 wt %, at least about 10 wt %.

Suitably, the pectin (or other water-soluble enteric polymer) is present in the capsule shell in an amount of about 30 wt % or less, about 25 wt % or less, or about 20 wt % or less.

Suitably, the pectin (or other water-soluble enteric polymer) is present in the capsule shell in an amount of from about 3 wt % to about 30 wt %, from about 5 wt % to about 25 wt %, or about 10 wt % to about 20 wt %.

### Other Components

The capsule shell used in the present invention may comprise one or more further components such as a coagulants, moisture, colorants/opacifiers, plasticizers, or flavouring agents.

Suitably, the capsule shell used in the present invention comprises one or more coagulants. As used herein a “coagulant” may refer to agents which aid the formation of the film from the water-soluble film forming polymer. Exemplary coagulants include gellan gum, carrageenan, alginate, agar, konjac gum, locust beam gum. Suitably, the coagulant has a molecular weight of from about 450 kDa to about 550 kDa.

Suitably, the capsule shell used in the present invention further comprises gellan gum and/or carrageenan.

Suitably, the capsule shell used in the present invention comprises gellan gum and/or carrageenan (or other coagulant) in an amount of from about 0.5 wt % to about 3 wt %.

Suitably, the capsule shell used in the present invention does not comprise a coagulant. Suitably, the capsule shell used in the present invention does not comprise gellan gum and/or carrageenan. Suitably, the capsule shell used in the present invention does not comprise gellan gum.

Suitably, the capsule shell used in the present invention has a moisture content of at least about 2 wt %, at least about 3 wt %, or at least about 4 wt %. Suitably, the capsule shell used in the present invention has a moisture content of at least about 8 wt % or less, about 7 wt % or less, or about 6 wt % or less. Suitably, the capsule shell used in the present invention has a moisture content of from about 2 wt % to about 8 wt %, from about 3 wt % to about 7 wt %, from about 4 wt % to about 7 wt %, or from about 4 wt % to about 6 wt %.

Suitably, the capsule shell used in the present invention comprises one or more colorants and/or opacifiers. Suitable colorants and opacifiers will be known to those of skill in the art and include e.g. iron pigments, titanium dioxide, caramel, riboflavin, carmine, sodium copper, and chlorphyllin.

Suitably, the capsule shell used in the present invention comprises one or more plasticizers. Suitable plasticizers will be known to those of skill in the art and may include glycerol, PEG400, PEG1000 and triacetin (see e.g. Saringat, H. B., et al., 2005. Pak J Pharm Sci, 18(3), pp. 25-38). Suitably, the capsule shell may comprise one or plasticizers in an amount of from about 0 wt % to about 10 wt %, from about 0 wt % to about 5 wt %, or about 5 wt %.

Suitably, the capsule shell used in the present invention comprises glycerol. Suitably, the capsule shell may comprise glycerol in an amount of from about 0 wt % to about 10 wt %, from about 0 wt % to about 5 wt %, or about 5 wt %.

Suitably, the capsule shell used in the present invention comprises one or more flavouring agents. Suitable flavouring agents will be known to those of skill in the art.

Suitably, the capsule shell used in the present invention comprises, essentially consists of, or consists of:
(i) HPMC, in an amount of from about 65 wt % to about 97 wt %;
(ii) pectin, in an amount of from about 3 wt % to about 30 wt %; and
(iii) a moisture content of about 4 wt % to about 7 wt %.

In this context, “essentially consists of” may mean that no further water-soluble film forming polymers, no further water-soluble enteric polymers, and no coagulants are present in the capsule shell, but that other components, such as colorants/opacifiers, plasticizers, or flavouring agents, are not excluded.

### Method of Manufacturing Capsule Shell

Suitable methods of manufacturing the capsule shell used in the present invention will be known to the skilled person (see e.g. Majee, S. B., et al., 2017. Int J Pharm Pharm Sci, 9(10), pp. 1-6). Any commercially available capsule making machine may be used in the method of manufacturing.

Typically, a method of manufacturing a capsule shell comprises:
(a) providing a capsule shell composition comprising a water-soluble film forming polymer (e.g. HPMC), a water-soluble enteric polymer (e.g. pectin), a gelling aid (e.g. a divalent cation), and optionally any other components;
(b) mixing the capsule shell composition with a solvent (e.g. water), thereby forming a capsule shell solution;
(c) dipping of mould pins in the capsule shell solution; and
(d) withdrawing of the mould pins, drying, and stripping, thereby forming the body portion and/or cap portion of the capsule shell.

Suitably, the capsule shell composition comprises HPMC (or other water-soluble film forming polymer) in an amount of from about 65 wt % to about 97 wt %, from about 70 wt % to about 95wt %, from about 75 wt % to about 90 wt %, or from about 80 wt % to about 85 wt %.

Suitably, the capsule shell composition comprises pectin (or other water-soluble enteric polymer) in an amount of from about 3 wt % to about 30 wt %, from about 5 wt % to about 25wt %, or about 10 wt % to about 20 wt %.

Suitably, the capsule shell composition comprises a gelling aid in an amount of from about 0.05 wt % to about 5 wt %, for example about 0.3 wt %.

As used herein, a “gelling aid” may refer to an agent which aids gelling during capsule shell formation, such as monovalent cations or divalent cations, or salts thereof. Exemplary gelling aids include potassium ions, sodium ions, calcium ions, magnesium ions, zinc ions, potassium chloride, sodium chloride, calcium chloride, and magnesium chloride. Divalent cations may act as gelling aids by forming ionic bridges between ionised carboxyl groups of the galacturonic acid in the pectin.

Suitably, the gelling aid is a divalent cation, or a salt thereof. Suitably, the gelling aid is a calcium ion, a magnesium ion, a zinc ion, or a salt thereof. Suitably, the gelling aid is a calcium ion or a magnesium ion, or a salt thereof. Suitably, the gelling agent is calcium chloride.

Suitably, the capsule shell composition used in the present invention comprises, essentially consists of, or consists of:
(i) HPMC, in an amount of from about 65 wt % to about 97 wt %;
(ii) pectin, in an amount of from about 3 wt % to about 30 wt %; and
(iii) a divalent cation, in an amount of from about 0.05 wt % to about 5 wt %.

Any suitable conditions may be used in the method of manufacturing. Suitably, the mixing is conducted at a temperature of from about 40° C. to about 100° C. (e.g. about 80° C.), for the capsule shell composition to be completely dissolved in the solvent. Suitably, the dipping is conducted at a temperature of from about 40° C. to about 100° C. (e.g. about 55° C.), for the film to be formed. Suitably, the drying is conducted at a temperature of from about 10° C. to about 80° C. (e.g. about 25° C.), for forming the body portion and/or cap portion.

### Proteases

The capsule of the present invention comprises one or more proteases.

Proteases (also known as peptidases or proteinases) are enzymes that catalyse proteolysis, breaking down proteins into smaller polypeptides or amino acids. Proteases catalyse this reaction by cleaving the peptide bonds within proteins by hydrolysis. Proteases can be classified into broad groups including: serine proteases, cysteine proteases, threonine proteases, aspartic proteases, glutamic proteases, metalloproteases, and asparagine peptide lyases. Sources of proteases include plants, animals, and microbes. Any protease which is suitable for oral administration may be used in the present invention.

Suitably, the one or more proteases are derived from plants and/or animals. Suitably, the one or more proteases are selected from the group consisting of: bromelain, trypsin, papain, chymotrypsin, pancreatin, serratiopeptidase, ficain, nattokinase, or any combination thereof. Suitably, the one or more proteases are selected from the group consisting of: bromelain, trypsin, papain, and chymotrypsin, or any combination thereof.

In some embodiments, the one or more proteases comprise or consist of bromelain and/or trypsin. In some embodiments, the one or more proteases comprise or consist of bromelain and trypsin.

The capsule of the present invention may comprise the one or more proteases in any suitable amount.

Suitably, the capsule comprises the one or more proteases in a total amount of at least about 50 mg, at least about 75 mg, at least about 100 mg, at least about 125 mg, at least about 150 mg, or at least about 200 mg.

Suitably, the capsule comprises the one or more proteases in a total amount of about 1000 mg or less, about 800 mg or less, about 600 mg or less, about 500 mg or less, about 400 mg or less, or about 300 mg or less.

Suitably, the capsule comprises the one or more proteases in a total amount of from about 50 mg to about 1000 mg, from about 75 mg to about 800 mg, from about 100 mg to about 600 mg, from about 125 mg to about 500 mg, from about 150 mg to about 400 mg, or from about 200 mg to about 300 mg.

Suitably, the capsule comprises the one or more proteases in a total amount of at least about 1000 FIP units, at least about 1500 FIP units, at least about 2000 FIP units, all least about 2500 FIP units, or at least about 3000 FIP units.

Suitably, the capsule comprises the one or more proteases in a total amount of about 12000 FIP units or less, about 10000 FIP units or less, about 8000 FIP units or less, about 6000 FIP units or less, or about 4000 FIP units or less.

Suitably, the capsule comprises the one or more proteases in a total amount of from about 1000 FIP units to about 12000 FIP units, from about 1500 FIP units to about 10000 FIP units, from about 2000 FIP units to about 8000 FIP units, from about 2500 FIP units to about 6000 FIP units, or from about 3000 FIP units to about 4000 FIP units.

### Bromelain

In some embodiments, the capsule of the present invention comprises bromelain.

Bromelain is an enzyme extract usually derived from the stems of pineapples, although it exists in all parts of the fresh pineapple. As used herein, the term “bromelain” may refer to either of two protease enzymes extracted from the plants of the family Bromeliaceae, or it may refer to a combination of those enzymes along with other compounds produced in an extract. Bromelain enzymes are called fruit bromelain (EC 3.4.22.33) and stem bromelain (EC 3.4.22.32). Sources of bromelain are known in the art (see e.g. Arshad, Z. I. M., et al., 2014.Applied microbiology and biotechnology, 98(17), pp. 7283-7297). Suitably, the bromelain is stem bromelain.

Suitably, the capsule comprises bromelain in an amount of at least about 50 mg, at least about 75 mg, at least about 100 mg, or at least about 125 mg.

Suitably, the capsule comprises bromelain in an amount of about 500 mg or less, about 400 mg or less, about 300 mg or less, or about 200 mg or less.

Suitably, the capsule comprises bromelain in an amount of from about 50 mg to about 500 mg, from about 75 mg to about 400 mg, from about 100 mg to about 300 mg, from about 100 mg to about 200 mg, or from about 125 mg to about 200 mg.

Suitably, the capsule comprises bromelain in an amount of about 150 mg, about 180 mg, about 225 mg, or about 270 mg.

Suitably, the capsule comprises bromelain in an amount of about 150 mg.

Suitably, the capsule comprises bromelain in an amount of at least about 400 FIP units, at least about 500 FIP units, at least about 600 FIP units, at least about 700 FIP units, or at least about 800 FIP units.

Suitably, the capsule comprises bromelain in an amount of about 3000 FIP units or less, about 2500 FIP units or les, about 2000 FIP units or less, about 1500 FIP units or less, or about 1000 or less.

Suitably, the capsule comprises bromelain in an amount of from about 400 FIP units to about 3000 FIP units, from about 500 FIP units to about 2500 FIP units, from about 600 FIP units to about 2000 FIP units, from about 700 FIP units to about 1500 FIP units, or from about 800 FIP units to about 1000 FIP units.

Suitably, the capsule comprises bromelain in an amount of about 900 FIP units or about 1350 FIP units.

Suitably, the capsule comprises bromelain in an amount of about 900 FIP units.

### Trypsin

In some embodiments, the capsule of the present invention comprises trypsin.

Trypsin (EC 3.4.21.4) is a serine protease found in the digestive system of many vertebrates. Trypsin is available in high quantity in pancreases (e.g. in porcine, bovine, or ovine pancreas) and can be purified easily.

Suitably, the capsule comprises trypsin in an amount of at least about 20 mg, at least about 30 mg, at least about 40 mg, at least about 50 mg, or at least about 60 mg.

Suitably, the capsule comprises trypsin in an amount of about 250 mg or less, about 200 mg or less, about 150 mg or less, about 100 mg or less, or about 80 mg or less.

Suitably, the capsule comprises trypsin in an amount of from about 20 mg to about 250 mg, from about 30 mg to about 200 mg, from about 40 mg to about 150 mg, from about 50 mg to about 100 mg, or from about 60 mg to about 80 mg.

Suitably, the capsule comprises trypsin in an amount of about 72 mg, about 96 mg, about 108 mg, or about 144 mg.

Suitably, the capsule comprises trypsin in an amount of about 72 mg.

Suitably, the capsule comprises trypsin in an amount of at least about 1000 FIP units, at least about 1500 FIP units, at least about 1750 FIP units, at least about 2000 FIP units, at least about 2250 FIP units, or at least about 2500 FIP units.

Suitably, the capsule comprises trypsin in an amount of about 10000 FIP units or less, about 8000 FIP units or less, about 6000 FIP units or less, about 5000 FIP units or less, about 4500 FIP units or less, or about 4000 FIP units or less.

Suitably, the capsule comprises trypsin in an amount of about 1000 FIP units to about 10000 FIP units, from about 1500 FIP units to about 8000 FIP units, from about 1750 FIP units to about 6000 FIP units, from about 2000 FIP units to about 5000 FIP units, from about 2250 FIP units to about 4500 FIP units, or from about 2500 FIP units to about 4000 FIP units.

Suitably, the capsule comprises trypsin in an amount of about 2880 FIP units or about 4320 FIP units.

Suitably, the capsule comprises trypsin in an amount of about 2880 FIP units.

Suitably, the capsule comprises bromelain in an amount of from about 50 mg to about 500 mg (e.g. about 150 mg) and trypsin in an amount of from about 20 mg to about 250 mg (e.g. about 72 mg).

Suitably, the capsule comprises bromelain in an amount of from about 400 FIP units to about 3000 FIP units (e.g. about 900 FIP units) and trypsin in an amount of from about 1000 FIP units to about 10000 FIP units (e.g. about 2880 FIP units).

### Flavonoids

The capsule of the present invention may further comprise one or more flavonoid.

Flavonoids (also known as bioflavonoids) are a class of polyphenolic secondary metabolites found in plants which may include flavonoids, isoflavonoids and neoflavonoids. Flavonoids, isoflavonoids and neoflavonoids are natural products derived from 2-phenylchromen-4-one (flavone), 3-phenylchromen-4-one and 4-phenylcoumarin, respectively (IUPAC. Compendium of Chemical Terminology, 2nd ed. (the “Gold Book”). Compiled by A. D. McNaught and A. Wilkinson. Blackwell Scientific Publications, Oxford (1997)). Flavonoids can be classified into different broad groups including anthocyanidins, chalcones, flavonols, flavanones, flavan-3-ols, flavanonols, flavones, and isoflavonoids. Any flavonoid which is suitable for oral administration may be used in the present invention (see e.g. Ververidis, F., et al., 2007. Biotechnology Journal: Healthcare Nutrition Technology, 2(10), pp. 1214-1234).

Flavonoids can be found in plants in glycoside-bound and free aglycone forms. Suitably, the one or more flavonoids are in either form. In some embodiments, the one or more flavonoids are one or more flavonoid glycosides.

Suitably, the one or more flavonoids are derived from plants. Suitably, the one or more flavonoids are selected from the group consisting of: rutin, quercetin, kaempferol, myricetin, fisetin, catechin, epicatechin, hydroxytyrosol, oleuropein, hesperidin, or any combination thereof.

Suitably, the one or more flavonoids comprises or consists of one or more flavonol, in glycoside-bound or free aglycone form, optionally a flavonol glycoside.

Suitably, the one or more flavonoids comprises or consists of quercetin, in glycoside-bound or free aglycone form, optionally a quercetin glycoside.

In some embodiments, the one or more flavonoids comprise or consist of rutin.

Suitably, the capsule comprises the one or more flavonoids in a total amount of at least about 50 mg, at least about 100 mg, at least about 125 mg, or at least about 150 mg.

Suitably, the capsule comprises the one or more flavonoids in a total amount of about 1000 mg or less, about 600 mg or less, about 400 mg or less, or about 300 mg or less.

Suitably, the capsule comprises the one or more flavonoids in a total amount of from about 50 mg to about 1000 mg, from about 100 mg to about 600 mg, from about 125 mg to about 400 mg, or from about 150 mg to about 300 mg.

Suitably, the capsule comprises the one or more flavonoids in a total amount of about 200 mg, about 250 mg, or about 300 mg.

Suitably, the capsule comprises the one or more flavonoids in a total amount of about 200 mg.

Suitably, the capsule comprises: one or more proteases in a total amount of from about 50 mg to about 1000 mg; and one or more flavonoids flavonoids in a total amount of from about 50 mg to about 1000 mg.

### Rutin

In some embodiments, the capsule of the present invention comprises rutin.

Rutin (also called rutoside, quercetin-3-rutinoside, sophorin, 3,3′,4′,5,7-pentahydroxyflavone-3-rhamnoglucoside) is a flavonol, abundantly found in plants, such as passion flower, buckwheat, tea, and apple. Chemically it is a glycoside comprising of flavonolic aglycone quercetin along with disaccharide rutinose (see e.g. Ganeshpurkar, A. and Saluja, A. K., 2017. Saudi pharmaceutical journal, 25(2), pp. 149-164).

Suitably, the capsule comprises rutin in an amount of at least about 50 mg, at least about 100 mg, at least about 125 mg, or at least about 150 mg.

Suitably, the capsule comprises rutin in an amount of about 1000 mg or less, about 600 mg or less, about 400 mg or less, or about 300 mg or less.

Suitably, the capsule comprises rutin in an amount of from about 50 mg to about 1000 mg, from about 100 mg to about 600 mg, from about 125 mg to about 400 mg, or from about 150 mg to about 300 mg.

Suitably, the capsule comprises rutin in an amount of about 200 mg, about 250 mg, or about 300 mg.

Suitably, the capsule comprises rutin in an amount of about 200 mg.

Suitably, the capsule comprises: bromelain in an amount of from about 50 mg to about 500 mg (e.g. about 150 mg); trypsin in an amount of from about 20 mg to about 250 mg (e.g. about 72 mg); and rutin in an amount of from about 50 mg to about 1000 mg (e.g. about 200 mg).

Suitably, the capsule comprises: bromelain in an amount of from about 400 FIP units to about 3000 FIP units (e.g. about 900 FIP units); trypsin in an amount of from about 1000 FIP units to about 10000 FIP units (e.g. about 2880 FIP units); and rutin in an amount of from about 50 mg to about 1000 mg (e.g. about 200 mg).

### Other Components

The capsule of the present invention may comprise one or more further components such as bulking agents, preservatives, stabilisers, binders, emulsifiers, solubilising agents (e.g. oils, fats, waxes, lecithins etc.), adsorbents, carriers, fillers, dispersing agents, wetting agents, processing aids, flowing agents, weighting agents, taste-masking agents, flavouring agents, and sweeteners. The capsule of the present invention may also contain one or more conventional pharmaceutical additives, adjuvants, excipients, or diluents.

Suitably, the capsule of the present invention may comprise one or more bulking agent. Any suitable bulking agent may be used, such as cellulose derivatives, insoluble fiber, rice flour or lactose. Any suitable amount of bulking agent may be used, for example, depending on the concentration of active agents. Suitably, the capsule contents comprise bulking agent in an amount of about 25 wt % or less. Suitably, the capsule contents comprise bulking agent in an amount of about 1 wt % or more, about 5 wt % or more, or about 10 wt % or more. Suitably, the capsule contents comprise bulking agent in an amount of from about 1 wt % to about 25 wt %, from about 5 wt % to about 25 wt %, or from about 10 wt % to about 25 wt %.

Suitably, the bulking agent comprises or consists of one or more cellulose derivative (see e.g. Mastropietro, D.J. and Omidian, H., 2013. Drug development and industrial pharmacy, 39(2), pp. 382-392). Suitably, the cellulose derivative is selected from the group consisting of: powdered and microcrystalline cellulose (MCC), ethyl cellulose, hydroxypropyl cellulose (HPC), hydroxyethyl cellulose (HEC), hydroxypropyl methylcellulose (HPMC), HPMC phthalate, HPMC acetate succinate, cellulose acetate (CA), CA phthalate, sodium (Na) and calcium (Ca) carboxymethylcellulose (CMC), croscarmellose sodium (XCMCNa), methyl cellulose, and low substituted HPC, or any combination thereof.

Suitably, the one or more bulking agent comprises or consists of microcrystalline cellulose (MCC) and/or hydroxypropyl cellulose (HPC).

Suitably, the capsule of the present invention may comprise one or more preservative. Any suitable preservative may be used, such as an antioxidant. Suitably, the capsule of the present invention comprises L-ascorbyl palmitate. Suitably, the capsule contents comprise one or more preservative (e.g. an antioxidant) in an amount of less than about 1 wt %.

Suitably, the capsule of the present invention comprises one or more de-dusting agent. As used herein a “de-dusting agent” may refer to a de-dusting material which can be used to de-dust a granular enzyme preparation. Any suitable de-dusting agent may be used. Suitably, the capsule of the present invention comprises medium chain triglycerides. Suitably, the capsule contents comprise one or more de-dusting agent (e.g. medium chain triglycerides) in an amount of less than about 1 wt %.

Suitably, the capsule contents comprise or essentially consist of:
(i) bromelain, in an amount of from about 50 mg to about 500 mg (e.g. about 150 mg) or from about 400 FIP units to about 3000 FIP units (e.g. about 900 FIP units);
(ii) trypsin, in an amount of from about 20 mg to about 250 mg (e.g. about 72 mg) from about 1000 FIP units to about 10000 FIP units (e.g. about 2880 FIP units); and
(iii) rutin in an amount of from about 50 mg to about 1000 mg (e.g. about 200 mg).

In this context, “essentially consists of” may mean that no further active agents are encapsulated in the capsule shell, but that other components such as bulking agents and preservatives are not excluded.

### Release Profile

The capsule of the present invention may prevent release of the one or more proteases in the gastric phase and release the one or more proteases in the small intestinal phase, thereby mimicking the kinetic profile of an enterically coated tablet.

The “gastric phase” may refer to the period in which the capsule is in the stomach following oral administration. The “small intestinal phase” may refer to the period in which the capsule is in the small intestine following oral administration.

In vitro assays to simulate the gastric phase and small intestinal phase are known in the art (see e.g. Van de Wiele, T., et al., 2015. The impact of food bioactives on health, pp. 305-317) and may be used to determine the kinetic profile of the capsule of the present invention. In the context of an in vitro assay, the “gastric phase” may refer to gastric conditions for about 45minutes. Suitably, the gastric phase may comprise a pH of about 2.0 for 45 minutes at 37° C. In the context of an in vitro assay, the “small intestinal phase” may refer to small intestinal conditions for about 3 hours. Suitably, the small intestine phase may comprise: (i) a pH increase from about 5.5 to about 6.5 during the first hour at 37° C., (ii) a pH increase from about 6.5 to about 7.0 during the second hour at 37° C.; and (iii) and a constant pH of about 7.0 during the third hour at 37° C. Suitably, a standardized enzyme and bile liquid mix may be added in the small intestine phase. Suitably, the gastric phase and/or small intestinal phase may be simulated as described in the examples.

The capsule may remain intact in the gastric phase. Suitably, the capsule remains substantially intact in the gastric phase. In this context, “substantially intact” may mean that the capsule has minor damage, but that all product is still in the capsule, as determined by a visual inspection. Suitably, the capsule remains fully intact in the gastric phase. In this context, “fully intact” may mean that the capsule has not been damaged, as determined by a visual inspection

The capsule may be degraded in the small intestinal phase. Suitably, the capsule is substantially degraded in the small intestinal phase. In this context, “substantially degraded” (or “substantially dissolved”) may mean that the capsule has been damaged and all or substantially all product is released, as determined by a visual inspection. Suitably, the capsule is fully degraded in the small intestinal phase. In this context, “fully degraded” (or “fully dissolved”) may mean that the capsule has been completely destroyed, as determined by a visual inspection.

The capsule contents may not be released in the gastric phase. Suitably, protease activity is not substantially released in the gastric phase. In this context, “not substantially released” may mean that about 10% or less, about 5% or less, or about 1% or less of the protease activity present in the capsule is released. Suitably, protease activity is not released in the gastric phase. In this context, “not released” may mean that protease activity is not detectable in the gastric phase (e.g. that protease activity is not detected or that protease activity is not significantly greater than background protease activity).

The capsule contents may be released in the small intestinal phase. Suitably, protease activity is substantially released in the small intestinal phase. In this context, “substantially released” may mean that at least about 50%, at least about 60%, at least about 70%, at least about 80%, or at least about 90% of protease activity present in the capsule is released. Suitably, protease activity is fully released in the small intestinal phase. In this context, “fully released” may mean about 90% or more of protease activity present in the capsule is released.

The protease activity may be determined by any suitable assay. For example, the assays described in the example. Suitably, trypsin activity is determined using the TAME method. Suitably, bromelain activity is determined using the Z-RR-pNA method.

### Dosage and Size

Suitably, the capsule is formulated to provide an effective amount of the one or more active agents.

The capsule may be formulated to provide all or part of a daily dose. Suitable daily doses are described herein.

Suitably, the capsule is formulated to comprise about ½ a daily dose, about ⅓ of a daily dose, about ¼ of a daily dose, about ⅕ of a daily dose, or about ⅙ of a daily dose. Suitably, the capsule is formulated to comprise about ½ a daily dose, about ⅓ of a daily dose, about ¼ of a daily dose, or about ⅕ of a daily dose.

In some embodiments, the capsule is formulated to comprise about ½ a daily dose, or about ⅓ of a daily dose. In some embodiments, the capsule is formulated to comprise about ⅓ of a daily dose.

The capsule may be formulated to be any size suitable for oral administration. Suitably, the capsule size is 00, 0E, or 0. In preferred embodiments, the capsule size is 00. Suitably, the mass of the capsule (excluding the capsule shell) is about 500 mg to about 800 mg. In some embodiments, the mass of the capsule (excluding the capsule shell) is about 700 mg to about 750 mg. In other embodiments, the mass of the capsule (excluding the capsule shell) is about 500 mg to about 600 mg. In some embodiments, the capsule size is 00 and the mass of the capsule (excluding the capsule shell) is about 700 mg to about 750 mg. In other embodiments, the capsule size is 0 and the mass of the capsule (excluding the capsule shell) is about 500 mg to about 600 mg.

### Method of Manufacture

In one aspect, the present invention provides a method of manufacturing a capsule comprising one or more proteases. The method may comprise:
(a) providing a capsule shell comprising a water-soluble film forming polymer and a water-soluble enteric polymer; and
(b) filling the capsule shell with a capsule composition comprising one or more proteases.

The capsule shell may be any capsule shell described herein. For example, the capsule shell may comprise, essentially consist of, or consist of: (i) HPMC, in an amount of from about 65wt % to about 97 wt %; (ii) pectin, in an amount of from about 3 wt % to about 30 wt %; and (iii) a moisture content of about 4 wt % to about 7 wt %. The method may further comprise manufacturing the capsule shell, for example by any method described herein. Preferably, the capsule shell is acid resistant. More preferably, the capsule shell is gastro resistant.

The capsule composition may comprise any suitable components described herein. For example, the capsule composition may comprise (i) bromelain, in an amount of from about 50 mg to about 500 mg (e.g. about 150 mg) or from about 400 FIP units to about 3000 FIP units (e.g. about 900 FIP units); (ii) trypsin, in an amount of from about 20 mg to about 250 mg (e.g. about 72 mg) from about 1000 FIP units to about 10000 FIP units (e.g. about 2880 FIP units); and (iii) rutin in an amount of from about 50 mg to about 1000 mg (e.g. about 200 mg).

Any suitable process for filling the capsule shell may be used (see e.g. Murachanian, D., 2010. Journal of GXP Compliance, 14(3), pp. 31-42). Suitable capsule filling machines are commercially available (see e.g. Small, L. E. and Augsburger, L. L., 1977. Journal of pharmaceutical sciences, 66(4), pp. 504-509). Typically, a process for filling hard capsules will comprise: (a) separating the body portion and the cap portion; (b) filling the body portion with the capsule composition; (c) locking the body portion and the cap portion together; (d) ejecting the formed capsule.

In one aspect, the present invention provides a capsule obtained or obtainable by said method of manufacture.

### Pack of Capsules

In one aspect, the present invention provides a pack comprising a plurality of capsules according to the present invention.

Any suitable packaging may be used, for example blistering or bottling. Suitably, the plurality of capsules are packaged in a bottle or container. The plurality of capsules may be packaged under sterile conditions. The pack may comprise suitable labelling and/or instructions for oral administration.

Suitably, the pack comprises at least about 15 days supply, at least about 30 days supply, at least about 45 days supply, at least about 60 days supply, or at least about 90 days supply. Suitably, the pack comprises about 15 days supply, about 30 days supply, about 45 days supply, about 60 days supply, or about 90 days supply. Suitably, the pack comprises about 30 days supply.

Suitably, the pack comprises at least about 45 capsules, at least about 90 capsules, at least about 135 capsules, at least about 180 capsules, or at least about 270 capsules. Suitably, the pack comprises about capsules, about 90 capsules, about 135 capsules, about 180 capsules, or about 270 capsules. Suitably, the pack comprises about 90 capsules.

### Supplements and Medicaments

Oral administration of the capsule of the present invention may be associated with beneficial effects.

Oral administration of proteases may be associated with beneficial immunomodulatory effects. One possible mechanism is by regulation of T cell activation thresholds. At sites of inflammation, with vascular permeability locally increased, serum protein exudate can enter the interstitial compartment. Thus, trypsin and other proteases, along with complement components and IgM, are delivered and enriched at the site of inflammation. Proteases, such as trypsin, may cleave antigens which promote T-cell activation, thereby modulating the immune response (see e.g. Lehmann, P.V., 1996. Nephrology Dialysis Transplantation, 11(6), pp. 953-955).

Further, oral administration of rutin has been explored for a number of pharmacological effects, including immunomodulatory effects (see e.g. Ganeshpurkar, A. and Saluja, A.K., 2017. Saudi pharmaceutical journal, 25(2), pp. 149-164).

In one aspect, the present invention provides use of a capsule according to the present invention as a supplement. Suitably, the supplement is a nutritional supplement.

In one aspect, the present invention provides a capsule according to the present invention for use as a medicament.

In one aspect, the present invention provides a method of treatment comprising administering a capsule according to the present invention to a subject in need thereof.

In one aspect, the present invention provides use of one or more proteases in the manufacture of a medicament, wherein the medicament is in the form of a capsule according to the present invention.

In one aspect, the present invention provides a capsule according to the present invention for use in treating and/or preventing an inflammatory disease, joint pain, arthrosis, injuries, back pain, or sciatica, or for use in accelerating healing.

In one aspect, the present invention provides a method of treating and/or preventing an inflammatory disease, joint pain, arthrosis, injuries, back pain, or sciatica, or of accelerating healing, the method comprising administering a capsule according to the present invention to a subject in need thereof.

In one aspect, the present invention provides use of one or more proteases in the manufacture of a medicament for treating and/or preventing an inflammatory disease, joint pain, arthrosis, injuries, back pain, or sciatica, or for accelerating healing, wherein the medicament is in the form of a capsule according to the present invention.

### Inflammatory Disease

Oral administration of proteases may be associated with beneficial effects in treating and/or preventing inflammatory disease. For example, the oral administration of proteolytic enzymes has been shown to be a safe and effective option to treat acute thrombophlebitis in the absence of compression treatment (Baumueller, M. and Rau, S., 2018 Journal of Phlebology and Lymphology, 11(1), pp. 7-12). An enzyme combination of proteolytic enzymes has been shown to decrease and normalize the biomarkers of inflammation in vestibulodynia and painful bladder syndrome (Murina, F., et al, V., 2013. Open Journal of Obstetrics and Gynecology, 3(4A), 33232).

In one aspect, the present invention provides a capsule according to the present invention for use in treating and/or preventing an inflammatory disease.

In one aspect, the present invention provides a method of treating and/or preventing an inflammatory disease, the method comprising administering a capsule according to the present invention to a subject in need thereof.

In one aspect, the present invention provides use of one or more proteases in the manufacture of a medicament for treating and/or preventing an inflammatory disease, wherein the medicament is in the form of a capsule according to the present invention.

As used herein, an “inflammatory disease” may refer to a disease in which the immune system attacks the body's own tissues, resulting in inflammation. Exemplary inflammatory diseases include encephalitis, myelitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, spondylitis, periostitis, chondritis, nephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

In some embodiments, the inflammatory disease is selected from phlebitis, prostate inflammation, or cystitis.

### Joint Pain

Oral administration of proteases may be associated with beneficial effects in treating and/or preventing joint pain (see e.g. Wald, M. and Rovenský, J., 2017. Systemic Enzyme Therapy in Comprehensive Treatment of Degenerative Rheumatic Diseases in the Elderly. In Gerontorheumatology (pp. 363-366). Springer, Cham).

In one aspect, the present invention provides a capsule according to the present invention for use in treating and/or preventing joint pain.

In one aspect, the present invention provides a method of treating and/or preventing joint pain, the method comprising administering a capsule according to the present invention to a subject in need thereof.

In one aspect, the present invention provides use of one or more proteases in the manufacture of a medicament for treating and/or preventing joint pain, wherein the medicament is in the form of a capsule according to the present invention.

As used herein, “joint pain” or “arthralgia” may refer to aching or pain in the joints (e.g. without inflammation). The causes of arthralgia are varied and range, from a joints perspective, from degenerative and destructive processes such as osteoarthritis and sports injuries to inflammation of tissues surrounding the joints, such as bursitis. Affected joints may include those in knees, elbows, shoulders, hips, wrists, hands, and feet.

In some embodiments, the joint pain is joint pain in the elbow, joint pain in the fingers, joint pain in the knee, menopausal joint pain, or rheumatic pain.

### Arthrosis

Oral administration of proteases may be associated with beneficial effects in treating and/or preventing joint arthrosis. For example, in a randomized trial of subjects with moderate-to-severe knee osteoarthritis, oral administration of proteolytic enzymes were found to have comparable effectiveness to diclofenac in relieving pain and increasing function (Ueberall, M.A., et al., 2016. Journal of pain research, 9, pp. 941-961).

In one aspect, the present invention provides a capsule according to the present invention for use in treating and/or preventing arthrosis.

In one aspect, the present invention provides a method of treating and/or preventing arthrosis, the method comprising administering a capsule according to the present invention to a subject in need thereof.

In one aspect, the present invention provides use of one or more proteases in the manufacture of a medicament for treating and/or preventing arthrosis, wherein the medicament is in the form of a capsule according to the present invention.

As used herein, “arthrosis” may include arthritis and other similar disorders. The term arthritis may include osteoarthritis, rheumatoid arthritis, gout and pseudo-gout, septic arthritis, ankylosing spondylitis, juvenile idiopathic arthritis, Still's disease, and psoriatic arthritis.

In one aspect, the present invention provides a capsule according to the present invention for use in treating and/or preventing osteoarthritis.

In one aspect, the present invention provides a method of treating and/or preventing osteoarthritis, the method comprising administering a capsule according to the present invention to a subject in need thereof.

In one aspect, the present invention provides use of one or more proteases in the manufacture of a medicament for treating and/or preventing osteoarthritis, wherein the medicament is in the form of a capsule according to the present invention.

Osteoarthritis is a type of degenerative joint disease that results from breakdown of joint cartilage and underlying bone. Osteoarthritis can affect the hands, lower back, neck, and weight-bearing joints such as knees, hips, and feet.

In some embodiments, the osteoarthritis is osteoarthritis in the hip, osteoarthritis in the knee, arthrosis in the metatarsophalangeal joint of the big toe, osteoarthritis in the finger, osteoarthritis in the foot, osteoarthritis of the ankle, Heberden and Bouchard nodes.

In some embodiments, the osteoarthritis is osteoarthritis in the knee.

### Injuries

Oral administration of proteases may be associated with beneficial effects in treating and/or preventing injuries. For example, there are reports that enzyme products have proven effective in treating sport injuries such as sprains, contusions and compression injuries (Pöttgen, K., Nutrition as a form of treatment. Sports Medicine Newspaper 2017).

In one aspect, the present invention provides a capsule according to the present invention for use in treating and/or preventing injuries.

In one aspect, the present invention provides a method of treating and/or preventing injuries, the method comprising administering a capsule according to the present invention to a subject in need thereof.

In one aspect, the present invention provides use of one or more proteases in the manufacture of a medicament for treating and/or preventing injuries, wherein the medicament is in the form of a capsule according to the present invention.

In some embodiments, the injuries are selected from sports injuries, muscle soreness, strains, overload damage, sprains, or bruises.

### Back pain and Sciatica

Oral administration of proteases may be associated with beneficial effects in treating and/or preventing back pain and sciatica.

In one aspect, the present invention provides a capsule according to the present invention for use in treating and/or preventing back pain or sciatica.

In one aspect, the present invention provides a method of treating and/or preventing back pain or sciatica, the method comprising administering a capsule according to the present invention to a subject in need thereof.

In one aspect, the present invention provides use of one or more proteases in the manufacture of a medicament for treating and/or preventing back pain or sciatica, wherein the medicament is in the form of a capsule according to the present invention.

In some embodiments, the back pain is selected from upper back pain, disc prolapse, or lumbago.

### Healing

Oral administration of proteases may be associated with beneficial effects in accelerating healing. For example, the percentage of patients with a severe Caesarean section scars scar was lower in a group treated with proteolytic enzymes (Dosedla, E., et al., 2016. Ceska gynekologie, 81(3), pp. 202-207).

In one aspect, the present invention provides a capsule according to the present invention for use in accelerating healing.

In one aspect, the present invention provides a method of accelerating healing, the method comprising administering a capsule according to the present invention to a subject in need thereof.

In one aspect, the present invention provides use of one or more proteases in the manufacture of a medicament for accelerating healing, wherein the medicament is in the form of a capsule according to the present invention.

In some embodiments, the gastro resistant capsule accelerates wound healing or healing after operation

### Administration

The gastro resistant capsule of the present invention may be orally administered in any suitable regime in order to deliver an effective amount of the one or more active agents.

Suitably, one, two, three, four, five, or six gastro resistant capsules are administered daily. Suitably, five or fewer, four or fewer, or three or fewer gastro resistant capsules are administered daily. In some embodiments, two or three gastro resistant capsules are administered daily. In some embodiments, three gastro resistant capsules are administered daily. The resistant capsules may be administered simultaneously or separately, or any combination thereof.

Suitably, the gastro resistant capsule is administered with at least about 200 ml of water.

Suitably, the gastro resistant capsule is administered in a fasting state. Suitably, the gastro resistant capsule is administered is administered at least about 45 minutes before a meal.

Suitably, the gastro resistant capsule is administered for at least 15 days, for at least 30 days, for at least 45 days, or for at least 60 days.

### Total Proteases

Suitably, the one or more proteases are administered in a total daily dose of at least about 100 mg, at least about 200 mg, at least about 300 mg, at least about 400 mg, at least about 500 mg, or at least about 600 mg.

Suitably, the one or more proteases are administered in a total daily dose of about 2000 mg or less, about 1500 mg or less, about 1200 mg or less, about 1000 mg or less, about 900 mg or less, or about 800 mg or less.

Suitably, the one or more proteases are administered in a total daily dose of from about 100 mg to about 2000 mg, from about 200 mg to about 1500 mg, from about 300 mg to about 1200 mg, from about 400 mg to about 1000 mg, from about 500 mg to about 900 mg, or from about 600 mg to about 800 mg.

Suitably, the one or more proteases are administered in a total daily dose of about 666 mg or about 929 mg.

Suitably, the one or more proteases are administered in a total daily dose of about 666 mg.

Suitably, the one or more proteases are administered in a total daily dose of at least about 2000 FIP units, at least about 4000 FIP units, at least about 6000 FIP units, at least about 8000 FIP units, or at least about 10000 FIP units.

Suitably, the one or more proteases are administered in a total daily dose of about 30000 FIP units or less, about 25000 FIP units or less, about 20000 FIP units or less, about 15000 FIP units or less, or about 12000 FIP units or less.

Suitably, the one or more proteases are administered in a total daily dose of from about 2000FIP units to about 30000 FIP units, from about 4000 FIP units to about 25000 FIP units, from about 6000 FIP units to about 20000 FIP units, from about 8000 FIP units to about 15000 FIP units, or from about 10000 FIP units to about 12000 FIP units.

Suitably, the one or more proteases are administered in a total daily dose of about 11340 FIP units.

### Bromelain

Suitably, bromelain is administered in a daily dose of at least about 100 mg, at least about 200 mg, at least about 300 mg, at least about 350 mg, at least about 400 mg, or at least about 450 mg.

Suitably, bromelain is administered in a daily dose of about 1000 mg or less, about 900 mg or less, about 800 mg or less, about 700 mg or less, about 600 mg or less, or about 500 mg or less.

Suitably, bromelain is administered in a daily dose of from about 100 mg to about 1000 mg, from about 200 mg to about 900 mg, from about 300 mg to about 800 mg, from about 350 mg to about 700 mg, from about 400 mg to about 600 mg, or from about 450 mg to about 500 mg.

Suitably, bromelain is administered in a daily dose of about 450 mg or about 540 mg.

Suitably, bromelain is administered in a daily dose of about 450 mg.

Suitably, bromelain is administered in a daily dose of at least about 500 FIP units, at least about 1000 FIP units, at least about 1500 FIP units, at least about 2000 FIP units, or at least about 2500 FIP units.

Suitably, bromelain is administered in a daily dose of about 10000 FIP units or less, about 8000 FIP units or less, about 6000 FIP units or less, about 4000 FIP units or less, or about 3000 FIP units or less.

Suitably, bromelain is administered in a daily dose of about 500 FIP units to about 10000 FIP units, from about 1000 FIP units to about 8000 FIP units, from about 1500 FIP units to about 6000 FIP units, from about 2000 FIP units to about 4000 FIP units, or from about 2500 FIP units to about 3000 FIP units.

Suitably, bromelain is administered in a daily dose of about 2700 FIP units.

### Trypsin

Suitably, trypsin is administered in a daily dose of at least about 50 mg, at least about 100 mg, at least about 150 mg, at least about 175 mg, or at least about 200 mg.

Suitably, trypsin is administered in a daily dose of about 500 mg or less, about 400 mg or less, about 350 mg or less, about 300 mg or less, or about 250 mg or less.

Suitably, trypsin is administered in a daily dose of from about 50 mg to about 500 mg, from about 100 mg to about 400 mg, from about 150 mg to about 350 mg, from about 175 mg to about 300 mg, or from about 200 mg to about 250 mg.

Suitably, trypsin is administered in a daily dose of about 216 mg or about 288 mg.

Suitably, trypsin is administered in a daily dose of about 216 mg.

Suitably, trypsin is administered in a daily dose of at least about 2000 FIP units, at least about 3000 FIP units, at least about 4000 FIP units, at least about 5000 FIP units, at least about 6000 FIP units or at least about 7000 FIP units.

Suitably, trypsin is administered in a daily dose of about 20000 FIP units or less, about 15000 FIP units or less, about 12000 FIP units or less, about 11000 FIP units or less, about 10000 FIP units or less, or about 9000 FIP units or less.

Suitably, trypsin is administered in a daily dose of from about 2000 FIP units to about 20000 FIP units, from about 3000 FIP units to about 15000 FIP units, from about 4000 FIP units to about 12000 FIP units, from about 5000 FIP units to about 11000 FIP units, from about 6000 FIP units to about 10000 FIP units, or from about 7000 FIP units to about 9000 FIP units.

Suitably, trypsin is administered in a daily dose of about 8640 FIP units.

### Total Flavonoids

Suitably, one or more flavonoids is administered in a total daily dose of at least about 100 mg, at least about 200 mg, at least about 300 mg, at least about 400 mg, or at least about 500 mg.

Suitably, one or more flavonoids is administered in a total daily dose of about 1200 mg or less, about 1000 mg or less, about 900 mg or less, about 800 mg or less, or about 700 mg or less.

Suitably, one or more flavonoids is administered in a total daily dose of from about 100 mg to about 1200 mg, from about 200 mg to about 1000 mg, from about 300 mg to about 900 mg, from about 400 mg to about 800 mg, from about 500 mg to about 700 mg.

Suitably, one or more flavonoids is administered in a total daily dose of about 600 mg.

Rutin

Suitably, rutin is administered in a total daily dose of at least about 100 mg, at least about 200 mg, at least about 300 mg, at least about 400 mg, or at least about 500 mg.

Suitably, rutin is administered in a total daily dose of about 1200 mg or less, about 1000 mg or less, about 900 mg or less, about 800 mg or less, or about 700 mg or less.

Suitably, rutin is administered in a total daily dose of from about 100 mg to about 1200 mg, from about 200 mg to about 1000 mg, from about 300 mg to about 900 mg, from about 400 mg to about 800 mg, from about 500 mg to about 700 mg.

Suitably, rutin is administered in a total daily dose of about 600 mg.

### Subject

The subject administered the gastro resistant capsule may be any subject in need thereof. For example the subject may have, or may be at risk of, any of the diseases or disorders described herein. The subject may be a human. The subject may be an adult.

Use of gastro resistant capsules

In one aspect, the present invention provides for use of a capsule shell to deliver one or more proteases. The gastro resistant capsule shell and the one or more proteases may be any described herein.

In one aspect, the present invention provides for use of gastro resistant capsule to deliver one or more proteases. The gastro resistant capsule and the one or more proteases may be any described herein.

The one or more proteases may be delivered to the small intestine (e.g. duodenum, jejunum and/or ileum). Suitably, the one or more proteases may be delivered to the duodenum.

### EXAMPLES

The invention will now be further described by way of examples, which are meant to serve to assist one of ordinary skill in the art in carrying out the invention and are not intended in any way to limit the scope of the invention.

### Example 1—release of trypsin and bromelain from different formulations using dynamic upper gastrointestinal tract simulation

### Materials and Methods

### Product Formulations

Four different product formulations, containing a mixture of trypsin and bromelain, were tested:
A tablet formulation, Wobenzym PS tablet.
A negative control HPMC capsule formulation, Wobenzym control HPMC capsule.
A gastro resistant HPMC capsule formulation where the capsule shell comprises gellan gum, Wobenzym HPMC/gellan gum capsule.
A gastro resistant HPMC capsule formulation where the capsule shell comprises pectin, Wobenzym HPMC/pectin capsule.

### Trypsin Activity Assay

To measure the trypsin activity, the standard TAME method was used. P-toluene-sulfonyl-L-arginine methyl ester (TAME) is a specific substrate for trypsin. One unit of trypsin hydrolyses 1 μmol of TAME per minute at pH 8.1 and 25° C. To evaluate the activity of the trypsin, samples were analysed by measuring the absorbance at 247 nm for 10 min every 50 seconds. Next, the trypsin activity of the samples was calculated using the equation below:

$$\frac{Units}{mL} = \frac{[ Δ A 247 sample - Δ A 247 blank ] \times 1000 \times 0.2}{540 \times Y}$$

### Where:

ΔA247=the slope of the initial linear portion of the curve;
Y=volume of the sample in the final reaction mixture (mL);
540=molar extinction coefficient of TAME at 247 nm (L/(mol×cm));
0.2=volume (in mL) of the reaction mixture.

The enzymatic activities were expressed in USP units/reactor, taking into account the volume of the reactor in each intestinal phase. The trypsin activity in TAME units/mL can be expressed as USP units/mL by incorporating a conversion factor of 19.2.

### Bromelain Activity Assay

To measure the bromelain activity, the method described in Hale, L. P., et al., (2005). International Immunopharmacology 5 (4), 783-793 was used, except that soybean trypsin inhibitor (STI) was added to inhibit the trypsin activity of the samples prior to the addition of the substrate and the actual measurement. This inhibition period lasted 5 min and was performed at 25° C.

One unit of bromelain hydrolyses 1 μmol of Z-Arg-Arg-pNA per minute at pH 7.3 and 25° C. To evaluate the activity of bromelain, the samples were analysed by measuring the absorbance at 410 nm for 15 min every 55 seconds. Next, the bromelain activity of the samples was calculated using the equation below:

$$\frac{Units}{mL} = \frac{[ Δ A 410 sample - Δ A 410 blank ] \times 1000 \times 0.2}{8000 \times Y}$$

### Where:

ΔA410=the slope of the initial linear portion of the curve;
Y=volume of the sample in the final reaction mixture (mL);
8000=molar extinction coefficient of pNA at 410 nm (L/(mol×cm));
0.2=volume (in mL) of the reaction mixture.

### Upper GIT Simulation

The product formulations were tested using an adapted SHIME® system representing the physiological conditions of the stomach and the small intestine within the same reactor over time. In order to mimic fed or fasted conditions, a specific gastric suspension is added to the reactor. After this, a standardized enzyme and bile liquid is added to simulate the small intestinal condition. The incubation conditions (i.e., pH profiles and incubation times) are optimized in order to resemble in vivo conditions in the different regions of the gastrointestinal tract for fasted or fed conditions.

The InfoGest consensus method (see Mackie, A., and Rigby, N. (2015). InfoGest Consensus Method. In ‘The Impact of Food Bioactives on Health’, Springer International Publishing, pp. 13-22) was used, except with a dynamic pH profile, which mimics the in vivo condition more closely. The applied conditions are summarised below.

### Gastric Phase (Fasted State):

Incubation during 45 min at 37° C., while mixing via stirring, pH=2.0.
Pepsin is supplied with the activity being standardized by measuring the absorbance increase at 280 nm of TCA-soluble products upon digestion of hemoglobin (reference protein).
Addition of phosphatidylcholine.
As background medium, only salts and mucins are supplied.
Sampling at t=15 min, 30 min, and 45 min.

### Small Intestinal Phase:

While mixing via stirring, the pH initially automatically increases from 2.0 to 5.5 within a period of 5 minutes after which the pH of the medium increases from 5.5 till 6.5 during the first hour, increases from 6.5 till 7.0 during the second hour and finally remains constant at pH 7.0 during the third hour. During the small intestinal phase, the temperature is set at 37° C. and controlled automatically by the software.
Regarding pancreatic enzymes, both a raw animal pancreatic extract (pancreatin) containing all the relevant enzymes in a specific ratio as well as defined ratios of the different enzymes are used.
Regarding bile salts, 3.33 mM bovine bile extract is generally supplemented (bovine bile is a closer match to human than porcine in terms of tauro-and glycocholate).

Sampling and visual scoring at t=30 min, 60 min, 90 min, 120 min, 150 min, and 180 min.

### Endpoints of the Upper GIT Simulation

At each sampling point, a visual inspection of the capsules/tablets was performed to study their dissolution behavior during passage through the different regions of the upper GIT. Scores were given on the basis of the following criteria:
1=capsule intact
2=capsule damaged but almost all product is still in capsule
3=capsule damaged and all product is released
4=capsule destroyed

The capsule/tablet scores at each sampling point are indicated above the bars in the figures.

### Statistics

Separate statistical analyses were performed for trypsin and bromelain and statistically significant differences between the enzymatic activities were determined in between each sampling point and its preceding one during the experiments under fasted conditions to demonstrate changes in function of time. In terms of statistics, the differences for all data discussed and indicated by “p<0.05” or “*” were significant with a confidence interval of 95%, as demonstrated using a paired Student's t-test.

### Results

Wobenzym PS Tablet

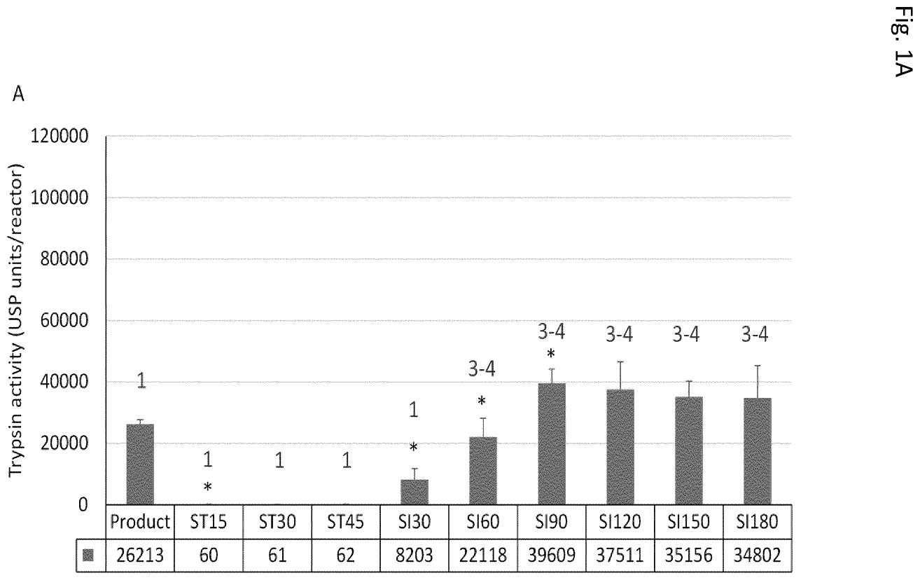

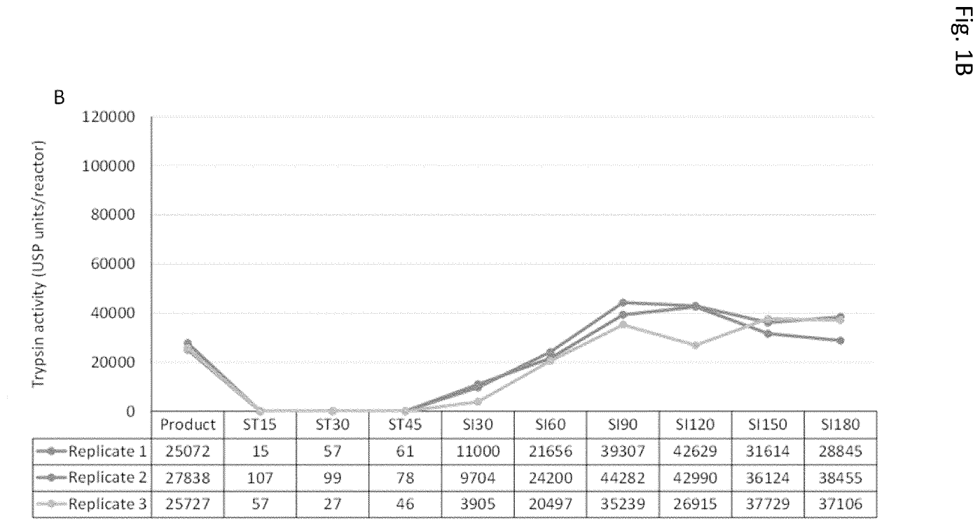

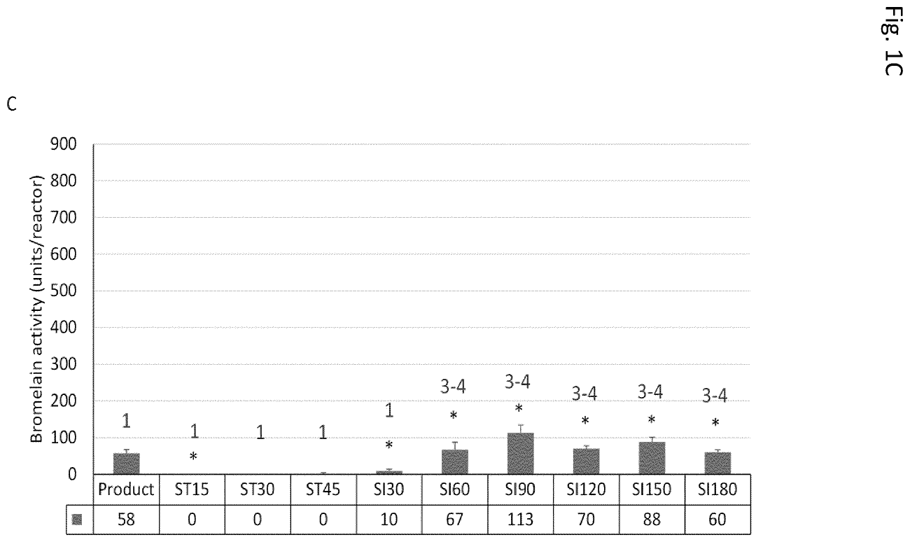

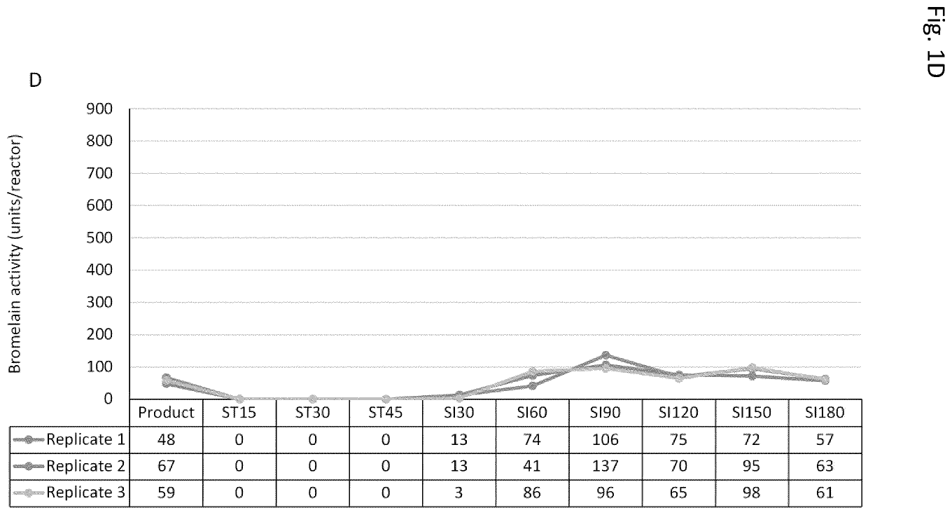

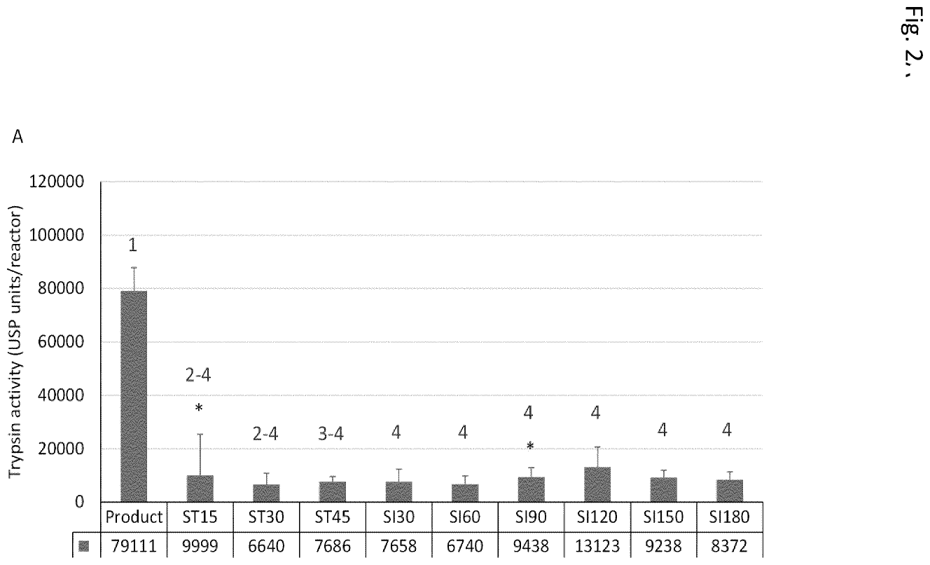

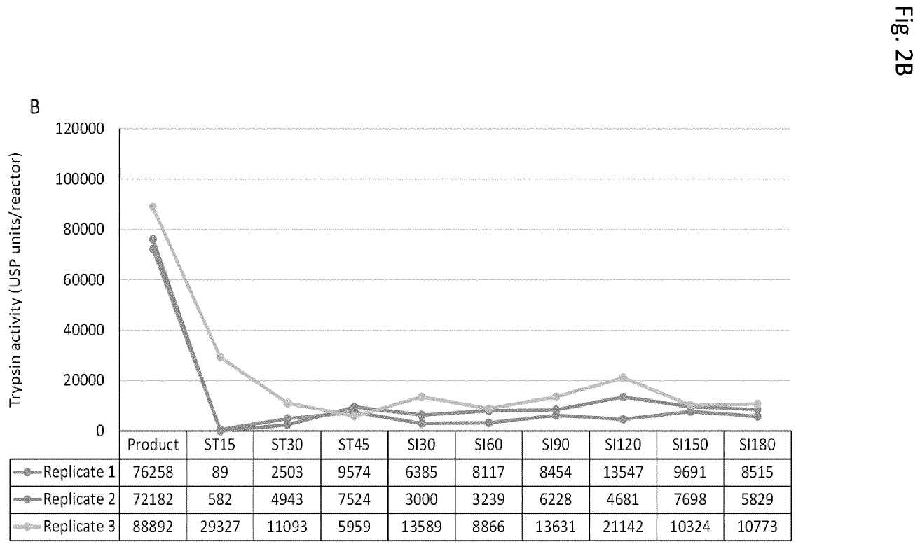

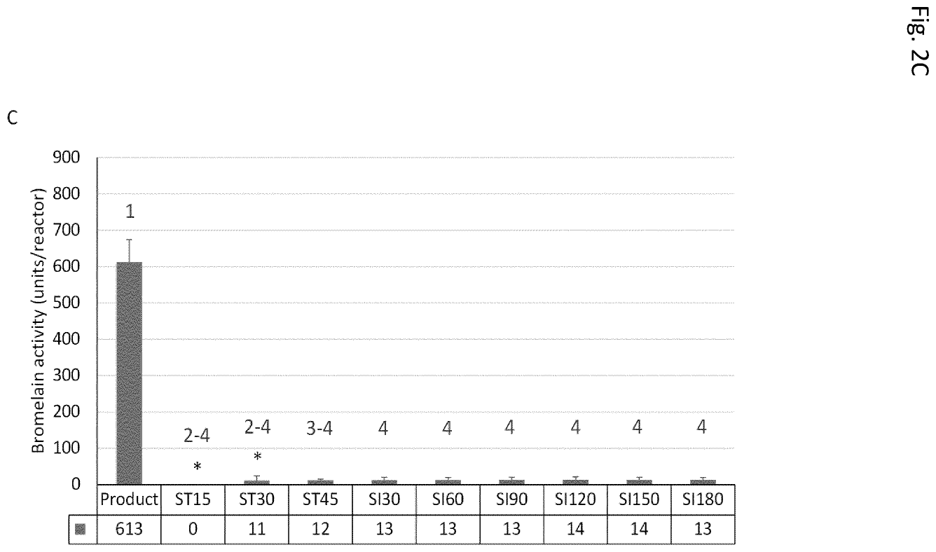

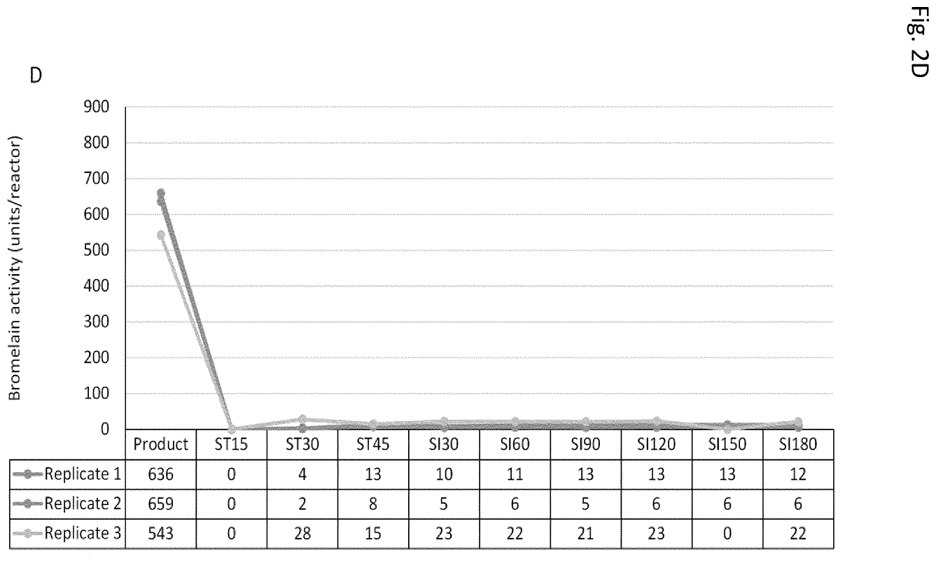

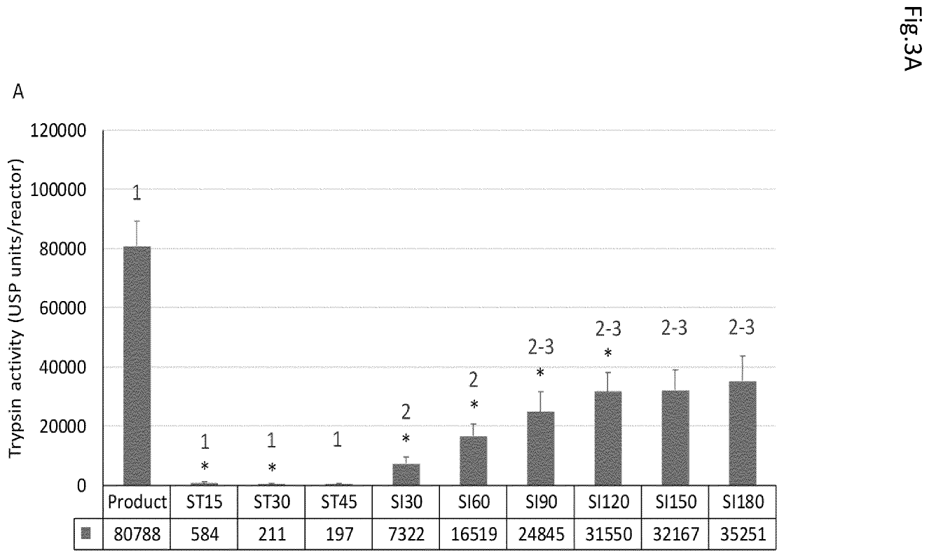

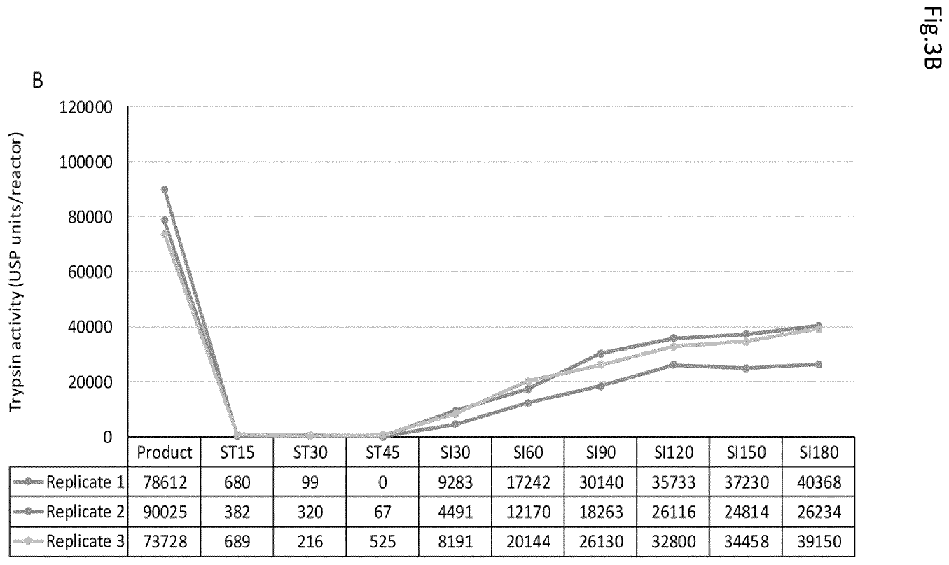

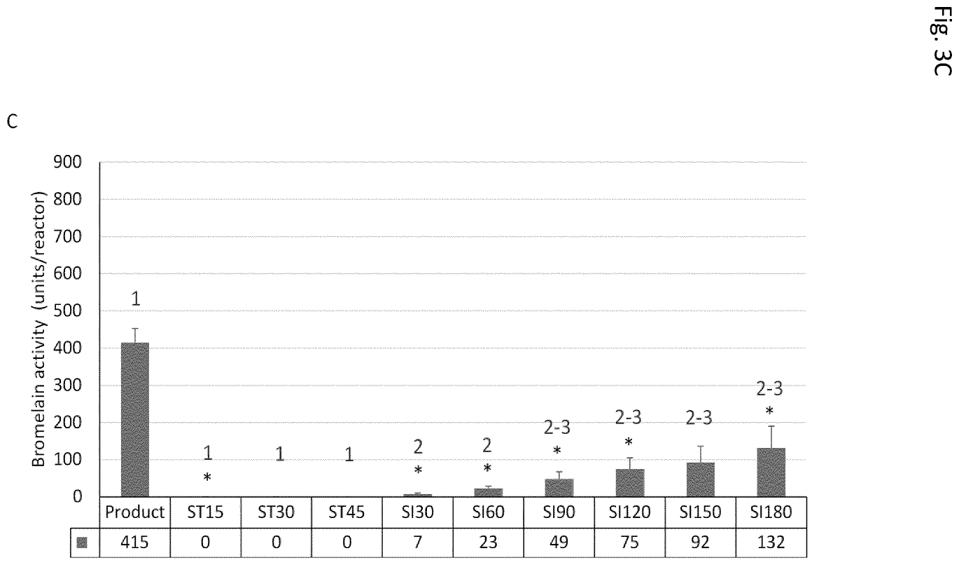

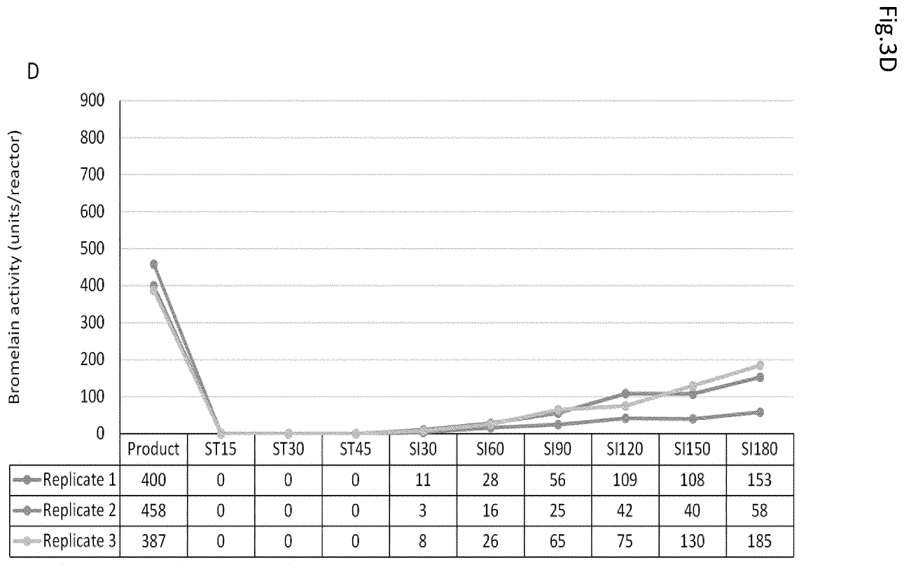

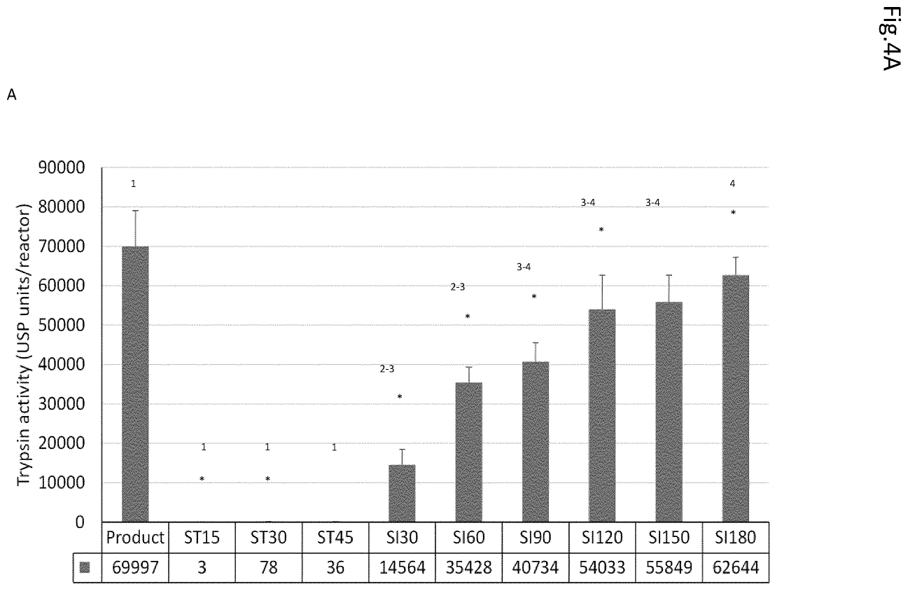

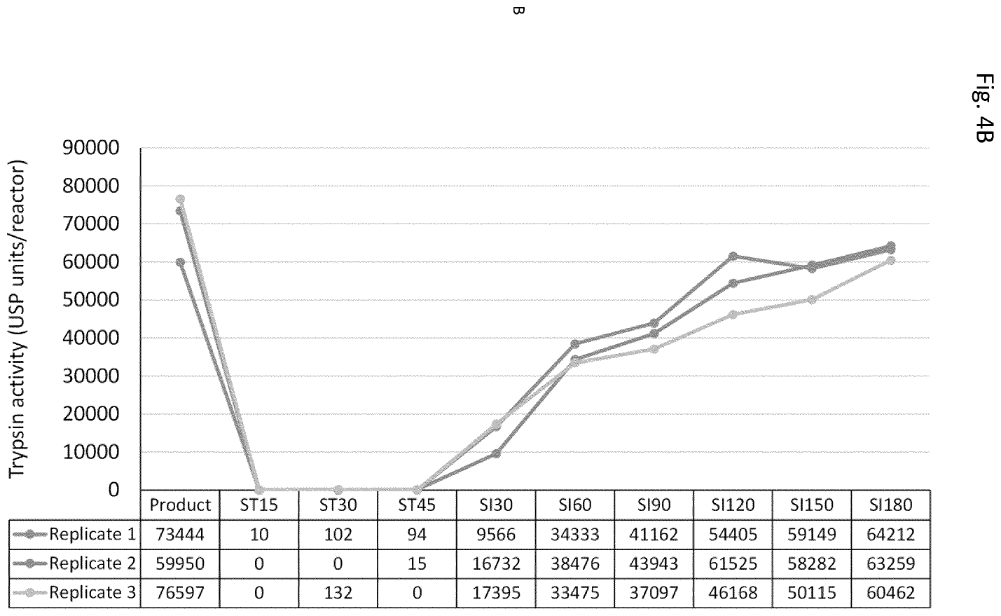

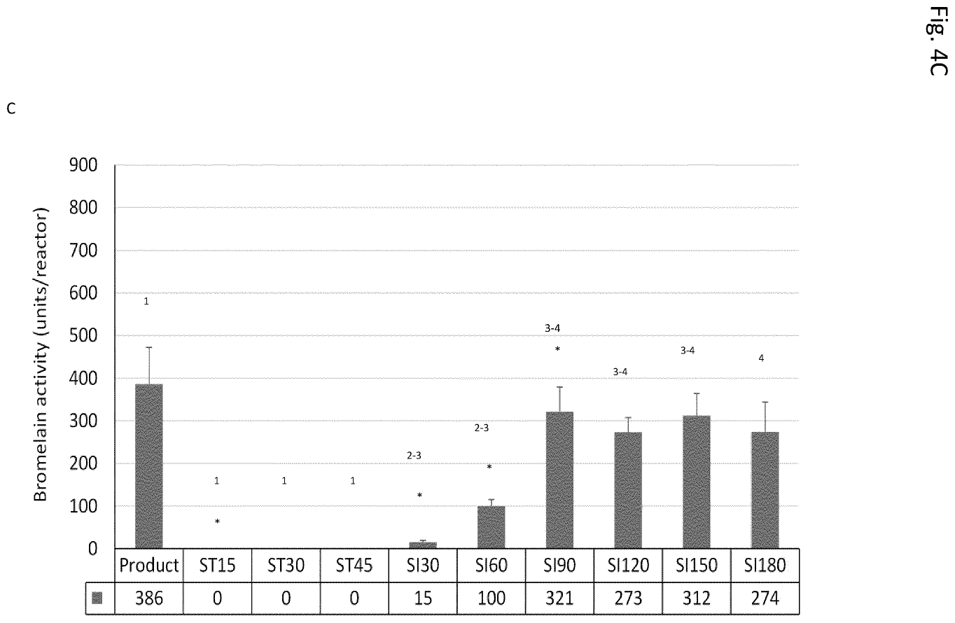

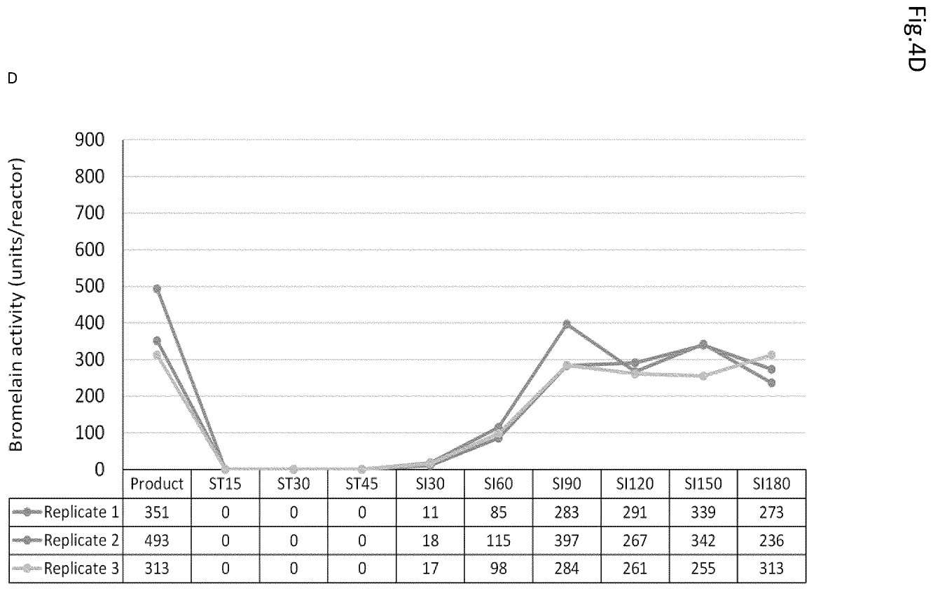

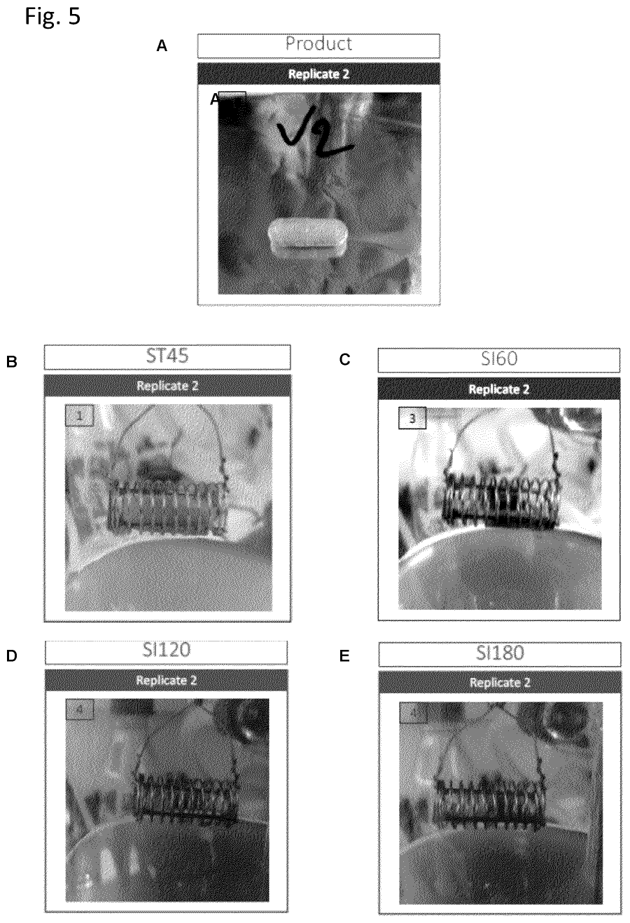

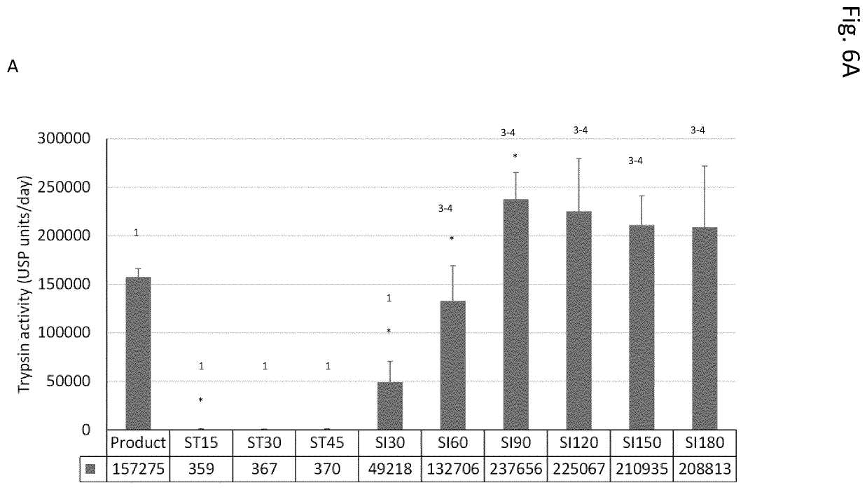

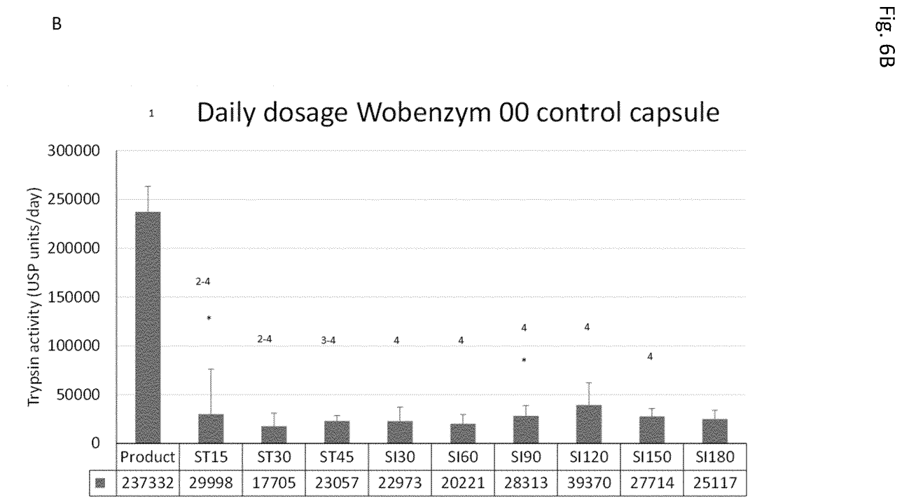

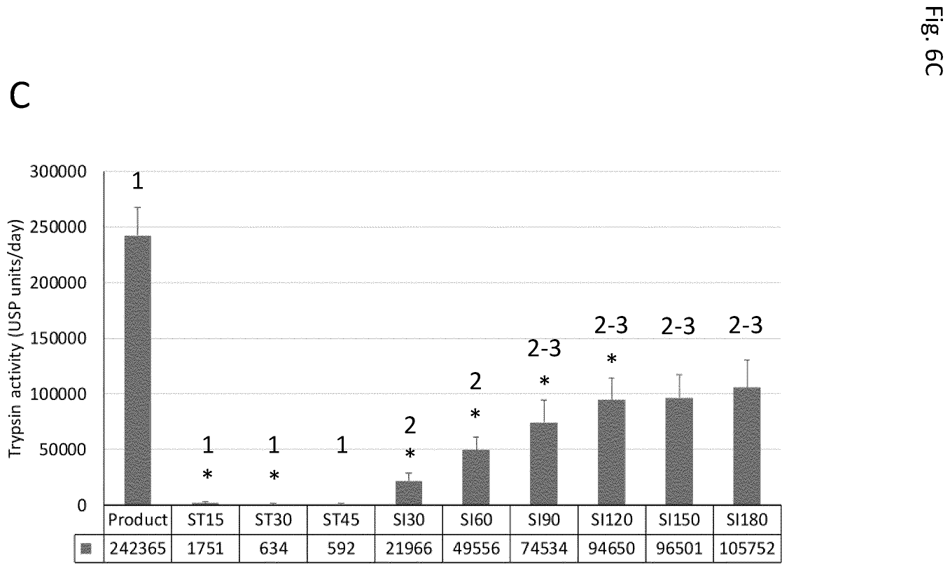

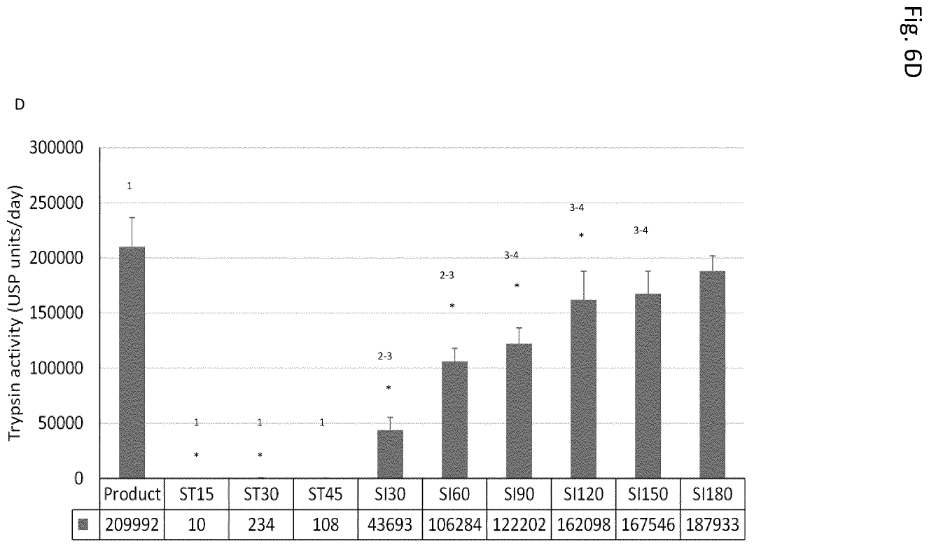

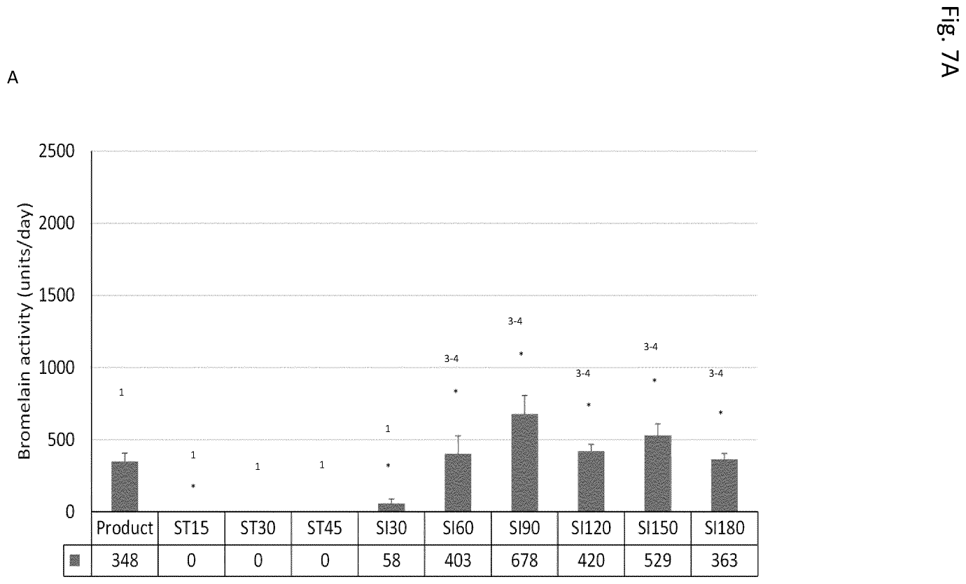

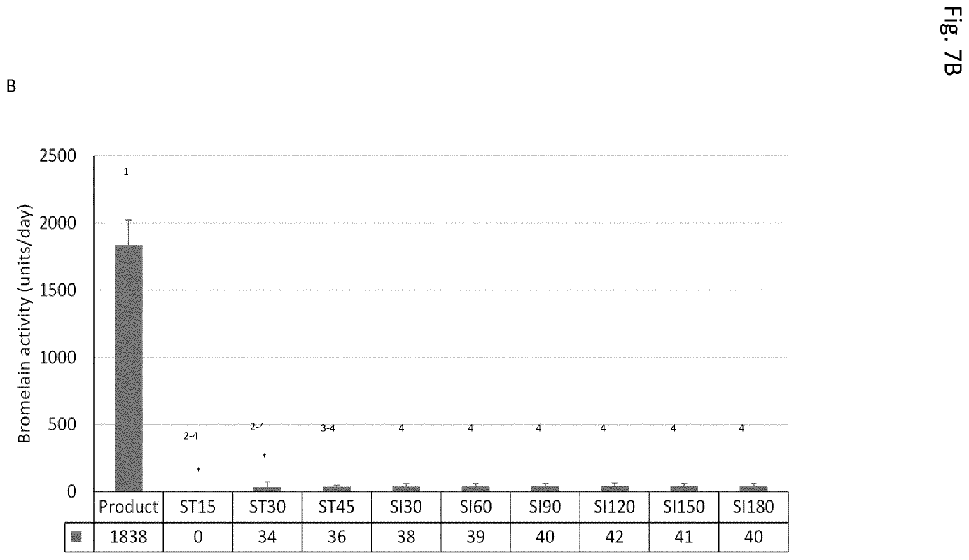

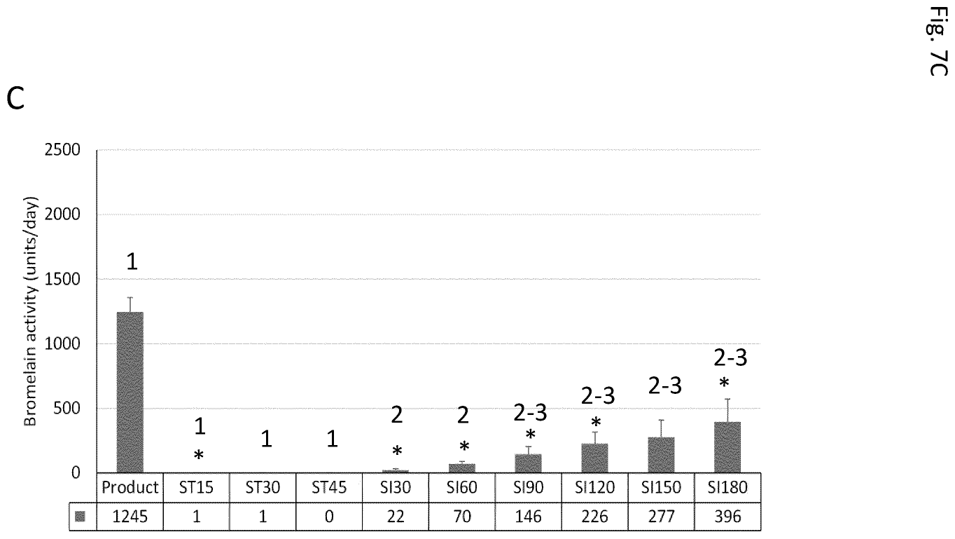

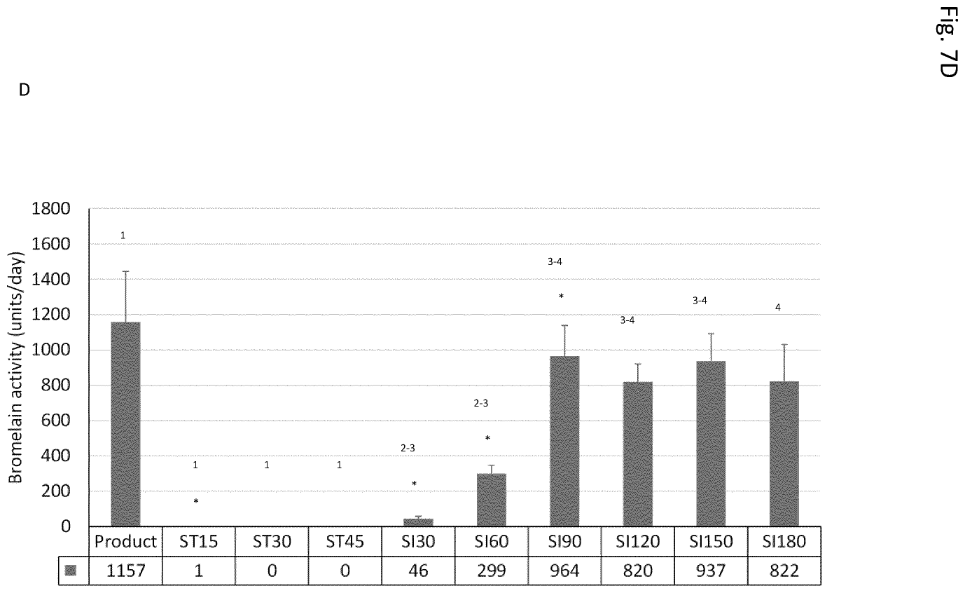

The activity of the released trypsin and bromelain from the Wobenzym PS tablet formulation along the upper GIT is presented in FIG. 1 (A-B: trypsin, C-D: bromelain). Both the average values (A and C) and the individual replicates (B and D) were reported.

First, it was observed that the intrinsic trypsin activity of the test product, i.e., the trypsin activity when the capsule content would be fully dissolved and not affected by the harsh environmental conditions of the stomach and small intestine, corresponded to 26,213 USP units/reactor, while the intrinsic bromelain activity corresponded to 58 units/reactor.

During the stomach phase, the tablet only disintegrated to a minor extent (capsule/tablet score of 1), which was accompanied by a very limited trypsin and even absent bromelain activity during gastric transit.

While no significant disintegration of the tablets was observed at the start of the small intestinal phase (capsule/tablet score of 1 at SI30), trypsin and bromelain activity significantly increased, indicating onset of release. Upon further incubation, tablets fully disintegrated, resulting in a maximum trypsin and bromelain activity observed after 90 min of small intestinal incubation. Afterwards, the activity of both enzymes remained unaffected, reaching an average activity of approximately 34,802 USP units/reactor for trypsin and 60 units/reactor for bromelain at the end of the small intestinal incubation.

These activities corresponded to the intrinsic trypsin and bromelain activity of the test product, indicating that the tablet formulation was able to protect trypsin and bromelain from the harsh environment encountered during upper gastrointestinal transit. This was probably achieved by the low exposure of trypsin and bromelain to the harsh pH conditions (pH of 2.0) encountered in the stomach phase, which will have protected the enzymes against denaturation and/or loss of activity.

### Wobenzym Control HPMC Capsule

The activity of the released trypsin and bromelain from the Wobenzym control HPMC capsule formulation along the upper GIT is presented in FIG. 2 (A-B: trypsin, C-D: bromelain). Both the average values (A and C) and the individual replicates (B and D) were reported.

First, it was observed that the intrinsic trypsin activity of the test product, i.e., the trypsin activity when the capsule content would be fully dissolved and not affected by the harsh environmental conditions of the stomach and small intestine, corresponded to 79,111 USP units/reactor, while the intrinsic bromelain activity corresponded to 613 units/reactor.

During the stomach phase, the control capsules were immediately damaged, resulting in (almost) full release of its content at the end of the gastric incubation. Even though the capsules completely disintegrated, trypsin and bromelain activity remained low. This can most likely be explained based on denaturation of the enzymes at the low pH-values encountered during the gastric phase. The denaturation and precipitation of the enzymes was also visually observed while performing the experiment since the released product was not fully dissolved.

During the small intestinal phase, enzymatic activity remained low and unaffected, indicating that the exposure of trypsin and bromelain to the harsh conditions encountered in the stomach phase strongly affected their biological activity.

### Wobenzym HPMC/Gellan Gum Vapsule

The activity of the released trypsin and bromelain from the Wobenzym HPMC/gellan gum capsule formulation along the upper GIT is presented in FIG. 3 (A-B: trypsin, C-D: bromelain). Both the average values (A and C) and the individual replicates (B and D) were included.

First, it was observed that intrinsic trypsin activity of the test product, i.e., the trypsin activity when the capsule content would be fully dissolved and not affected by the harsh environmental conditions of the stomach and small intestine, corresponded to 42, 100 USP units/reactor, while the intrinsic bromelain activity corresponded to 173 units/reactor.

During the stomach phase, only a very low trypsin and bromelain activity was detected. This was most likely the result from the fact that the capsules remained largely intact (capsule scores of 1-2), indicating that release of their content was rather limited.

However, during the small intestinal phase, the capsule score remained at 2 and therefore also the trypsin and bromelain activity remained rather low. Only a limited amount of the capsule content was thus released into the environment, resulting in an average trypsin and bromelain activity of 4,408 USP units/reactor and 5 units/reactor, respectively, at the end of the small intestinal phase.

As this is much lower than the trypsin and bromelain activity of the product as such, it can be concluded that this capsule formulation does not favour the release of a high amount of (active) trypsin and bromelain molecules along the upper GIT.

### Wobenzym HPMC/Pectin Capsule

The activity of the released trypsin and bromelain from the Wobenzym HPMC/pectin capsules along the upper GIT has been presented in FIG. 4 (A-B: trypsin, C-D: bromelain). Both the average values (A and C) and the individual replicates (B and D) were included. The pictures which were taken from the capsules were included in FIG. 5.

The average intrinsic trypsin activity of the test product was equal to 69,997 USP units/reactor, while the average intrinsic bromelain activity corresponded to 386 units/reactor.

During the gastric phase, only a very low trypsin activity was detected, and no bromelain activity was observed, which was the result of the capsules remaining (almost) entirely intact (i.e., the capsule score remained equal to 1 until ST45) and thus the corresponding very limited release of the capsule content into the environment. The exposure of the enzymes to the harsh environmental conditions encountered during the stomach phase was thus very limited.

During the small intestinal phase, further degradation of the capsules occurred. Full dissolution of the capsule was obtained during the final 30 minutes of the small intestinal phase, i.e., between SI150 and SI180. Consequently, the capsule content was gradually released into the environment, resulting in a gradual increase of the trypsin activity. By the end of the small intestinal phase, an average trypsin activity of 62,644 USP units/reactor was obtained, which is approximately 90% of the corresponding intrinsic activity. By the end of the small intestinal phase, the average bromelain activity had reached a maximum level and was equal to 274 units/reactor or approximately 71% of the corresponding intrinsic activity.

The dissolution behaviour of this specific capsule formulation resulted in a very high trypsin and bromelain activity by the end of the small intestinal phase, making this capsule formulation an ideal candidate to deliver the test enzymes at high concentrations at their site of activity.

### Comparison of Daily Dosage

In order to facilitate the comparison between the different product formulations, the daily required dosage was taken into account. The activity results were thereto multiplied with the number of capsules/tablets that would be administrated on a daily basis. These multiplication factors were as follows:
Wobenzym PS tablet=6 units/day
Wobenzym control HPMC capsule=3 units/day
Wobenzym HPMC/gellan gum capsule=3 units/day
Wobenzym HPMC/pectin capsule=3 units/day

The results are presented in FIGS. 6 and 7, for trypsin activity and bromelain activity, respectively. On a daily basis, the Wobenzym HPMC/pectin capsule proved to result in comparable enzymatic activity throughout the small intestinal transit compared to the Wobenzym PS tablet.

### EMBODIMENTS

Various preferred features and embodiments of the present invention will now be described with reference to the following numbered paragraphs (paras).
1. A gastro resistant capsule comprising one or more proteases, wherein the capsule shell comprises:
   (i) a water-soluble film forming polymer, in an amount of from about 65 wt % to about 97 wt %; and
   (ii) a water-soluble enteric polymer, in an amount of from about 3 wt % to about 30 wt %.
2. The gastro resistant capsule according to para 1, wherein the capsule shell is insoluble under gastric conditions and is soluble under small intestinal conditions.
3. The gastro resistant capsule according to para 2, wherein the gastric conditions are about pH 2.0 at about 37° C. and/or the small intestinal conditions are from about pH 5.5 to about pH 7.0 at about 37° C.
4. The gastro resistant capsule according to any preceding para, wherein the water-soluble enteric polymer is selected from one or more of pectin, propylene glycol alginate (PGA), or xanthum gum, preferably wherein the water-soluble enteric polymer is pectin.
5. The gastro resistant capsule according to para 4, wherein the pectin has a degree of esterification of from about 15% to about 40%, from about 20% to about 35%, or from about 20% to about 30%.
6. The gastro resistant capsule according to para 4 or 5, wherein the pectin has a degree of amidation of from about 0% to about 25%, from about 10% to about 25%, or from about 15% to about 25%.
7. The gastro resistant capsule according to any preceding para, wherein the water-soluble enteric polymer has a molecular weight of from about 20 kDa to about 1000 kDa, from about 40 kDa to about 400 kDa, or from about 50 kDa to about 200 kDa.
8. The gastro resistant capsule according to any preceding para, wherein the water-soluble film forming polymer is selected from one or more of hydroxypropyl methylcellulose (HPMC), starch, gelatin, pullulan, polyvinyl alcohol, hydroxypropylated starch, hydroxyethylated starch, hydroxypropyl cellulose, methylcellulose, hydroxyethyl cellulose, and hydroxyethyl methyl cellulose, preferably wherein the water-soluble film forming polymer is hydroxypropyl methylcellulose (HPMC).
9. The gastro resistant capsule according to any preceding para, wherein the water-soluble film forming polymer has a molecular weight of from about 50 kDa to about 815 kDa, or from about 50 kDa to about 400 kDa.
10. The gastro resistant capsule according to any preceding para, wherein the capsule shell further comprises a coagulant, such as gellan gum, carrageen, agar, konjac gum, or locust beam gum, in an amount of from about 0.5 wt % to about 3 wt %.
11. The gastro resistant capsule according to any of paras 1 to 9, wherein the capsule shell does not comprise a coagulant, preferably wherein the capsule shell does not comprise gellan gum and/or carrageenan.
12. The gastro resistant capsule according to any preceding para, wherein the capsule shell has a moisture content of from about 4 wt % to about 7 wt %.
13. The gastro resistant capsule according to any preceding para, wherein the one or more proteases are selected from the group consisting of: bromelain, trypsin, papain, pancreatin, chymotrypsin, serratiopeptidase, ficain, nattokinase, or any combination thereof.
14. The gastro resistant capsule according to any preceding para, wherein the one or more proteases comprise or consist of bromelain and/or trypsin.
15. The gastro resistant capsule according to any preceding para, wherein the one or more proteases comprise or consist of bromelain and trypsin.
16. The gastro resistant capsule according to any preceding para, wherein the gastro resistant capsule comprises the one or more proteases in a total amount of from about 50 mg to about 1000 mg, from about 75 mg to about 800 mg, from about 100 mg to about 600 mg, from about 125 mg to about 500 mg, from about 150 mg to about 400 mg, or from about 200 mg to about 300 mg.
17. The gastro resistant capsule according to any preceding para, wherein the gastro resistant capsule comprises the one or more proteases in a total amount of from about 1000 FIP units to about 12000 FIP units, from about 1500 FIP units to about 10000 FIP units, from about 2000 FIP units to about 8000 FIP units, from about 2500 FIP units to about 6000 FIP units, or from about 3000 FIP units to about 4000 FIP units.
18. The gastro resistant capsule according to any preceding para, wherein the gastro resistant capsule comprises bromelain in an amount of from about 50 mg to about 500 mg, from about 75 mg to about 400 mg, from about 100 mg to about 300 mg, or from about 125 mg to about 200 mg.
19. The gastro resistant capsule according to any preceding para, wherein the gastro resistant capsule comprises bromelain in an amount of about 150 mg.
20. The gastro resistant capsule according to any preceding para, wherein the gastro resistant capsule comprises bromelain in an amount of from about 400 FIP units to about 3000 FIP units, from about 500 FIP units to about 2500 FIP units, from about 600 FIP units to about 2000 FIP units, from about 700 FIP units to about 1500 FIP units, or from about 800 FIP units to about 1000 FIP units.
21. The gastro resistant capsule according to any preceding para, wherein the gastro resistant capsule comprises bromelain in an amount of about 900 FIP units.
22. The gastro resistant capsule according to any preceding para, wherein the gastro resistant capsule comprises trypsin in an amount of from about 20 mg to about 250 mg, from about 30 mg to about 200 mg, from about 40 mg to about 150 mg, from about 50 mg to about 100 mg, or from about 60 mg to about 80 mg.
23. The gastro resistant capsule according to any preceding para, wherein the gastro resistant capsule comprises trypsin in an amount of about 72 mg.
24. The gastro resistant capsule according to any preceding para, wherein the gastro resistant capsule comprises trypsin in an amount of from about 1000 FIP units to about 10000 FIP units, from about 1500 FIP units to about 8000 FIP units, from about 1750 FIP units to about 6000FIP units, from about 2000 FIP units to about 5000 FIP units, or from about 2500 FIP units to about 4000 FIP units.
25. The gastro resistant capsule according to any preceding para, wherein the gastro resistant capsule comprises trypsin in an amount of about 2880 FIP units.
26. The gastro resistant capsule according to any preceding para, wherein the gastro resistant capsule comprises one or more flavonoid, such as rutin, quercetin, kaempferol, myricetin, fisetin, catechin, epicatechin, hydroxytyrosol, oleuropein, hesperidin, or any combination thereof.
27. The gastro resistant capsule according to para 26, wherein the one or more flavonoid comprises or consists of rutin.
28. The gastro resistant capsule according to para 26 or 27, wherein the gastro resistant capsule comprises the one or more flavonoid in a total amount of from about 50 mg to about 1000 mg, from about 100 mg to about 600 mg, from about 125 mg to about 400 mg, or from about 150 mg to about 300 mg.
29. The gastro resistant capsule according to any of paras 26 to 28, wherein the gastro resistant capsule comprises the one or more flavonoid in a total amount of about 200 mg.
30. The gastro resistant capsule according to any preceding para, wherein the gastro resistant capsule further comprises one or more bulking agent.
31. The gastro resistant capsule according to para 30, wherein the one or more bulking agent comprises or consists of one or more cellulose derivative, such as powdered and microcrystalline cellulose (MCC), ethyl cellulose, hydroxypropyl cellulose (HPC), hydroxyethyl cellulose (HEC), hydroxypropyl methylcellulose (HPMC), HPMC phthalate, HPMC acetate succinate, cellulose acetate (CA), CA phthalate, sodium (Na) and calcium (Ca) carboxymethylcellulose (CMC), croscarmellose sodium (XCMCNa), methyl cellulose, and low substituted HPC, or any combination thereof.
32. The gastro resistant capsule according to para 30 or 31, wherein the one or more bulking agent comprises or consists of microcrystalline cellulose (MCC) and/or hydroxypropyl cellulose (HPC).
33. The gastro resistant capsule according to any preceding para, wherein the gastro resistant capsule further comprises one or more antioxidant, such as L-ascorbyl palmitate.
34. The gastro resistant capsule according to any preceding para, wherein the gastro resistant capsule remains substantially intact in the gastric phase and/or wherein the gastro resistant capsule is substantially dissolved in the small intestinal phase.
35. The gastro resistant capsule according to any preceding para, wherein the gastro resistant capsule remains fully intact in the gastric phase and/or wherein the gastro resistant capsule is fully dissolved in the small intestinal phase.
36. The gastro resistant capsule according to any preceding para, wherein the protease activity is not substantially released in the gastric phase and/or wherein the protease activity is substantially released in the small intestinal phase.
37. The gastro resistant capsule according to any preceding para, wherein the protease activity is not released in the gastric phase and/or wherein the protease activity is fully released in the small intestinal phase.
38. A method of manufacturing an gastro resistant capsule comprising one or more proteases, the method comprising:
   (a) providing a gastro resistant capsule shell comprising:
      (i) a water-soluble film forming polymer, in an amount of from about 65 wt % to about 97 wt %; and
      (ii) a water-soluble enteric polymer, in an amount of from about 3 wt % to about 30 wt %; and
   (b) filling the gastro resistant capsule shell with a capsule composition comprising one or more proteases.
39. A gastro resistant capsule obtained or obtainable by the method according to para 38.
40. A pack comprising a plurality of gastro resistant capsules according to any of paras 1 to 37 or para 39.
41. The pack according to para 40, wherein the pack comprises about 45 or more capsules, about 90 or more capsules, about 135 or more capsules, about 180 or more capsules, or about 270 or more capsules.
42. The pack according to para 40 or 41, wherein the pack comprises about 15 daily doses or more, about 30 daily doses or more, about 45 daily doses or more, about 60 daily doses or more, or about 90 daily doses or more.
43. Use of a gastro resistant capsule according to any of paras 1 to 37 or 39 as a nutritional supplement.
44. A gastro resistant capsule according to any of paras 1 to 37 or 39, for use as a medicament.
45. A gastro resistant capsule according to any of paras 1 to 37 or 39, for use in treating and/or preventing an inflammatory disease, joint pain, arthrosis, injuries, back pain, or sciatica, or for use in accelerating healing.
46. A gastro resistant capsule according to any of paras 1 to 37 or 39, for use in treating and/or preventing an inflammatory disease, such as phlebitis, prostate inflammation, or cystitis.
47. A gastro resistant capsule according to any of paras 1 to 37 or 39, for use in treating and/or preventing joint pain, such as joint pain in the shoulder, joint pain in the elbow, joint pain in the fingers, joint pain in the knee, menopausal joint pain, or rheumatic pain.
48. A gastro resistant capsule according to any of paras 1 to 37 or 39, for use in treating and/or preventing arthrosis, preferably arthritis, more preferably osteoarthritis, such as osteoarthritis in the hip, osteoarthritis in the knee, arthrosis in the metatarsophalangeal joint of the big toe, osteoarthritis in the finger, osteoarthritis in the foot, osteoarthritis of the ankle, or Heberden and Bouchard nodes.
49. A gastro resistant capsule according to any of paras 1 to 37 or 39, for use in treating and/or preventing injuries, such as sports injuries, muscle soreness, strains, overload damage, sprains, or bruises.
50. A gastro resistant capsule according to any of paras 1 to 37 or 39, for use in treating and/or preventing back pain, such as upper back pain, disc prolapse, or lumbago, or sciatica.
51. A gastro resistant capsule according to any of paras 1 to 37 or 39, for use in accelerating healing, such as wound healing or healing after operation.
52. The use according to para 43, or the gastro resistant capsule for use according to any of paras 44-51, wherein the one or more proteases are administered in a total daily dose of from about 100 mg to about 2000 mg, from about 200 mg to about 1500 mg, from about 300 mg to about 1200 mg, from about 400 mg to about 1000 mg, from about 500 mg to about 900 mg, or from about 600 mg to about 800 mg.
53. The use according to para 43 or 52, or the gastro resistant capsule for use according to any of paras 44-52, wherein the one or more proteases are administered in a total daily dose of about 666 mg.
54. The use according to any of paras 43 or 52-53, or the gastro resistant capsule for use according to any of paras 44-53, wherein the one or more proteases are administered in a total daily dose of from about 2000 FIP units to about 30000 FIP units, from about 4000 FIP units to about 25000 FIP units, from about 6000 FIP units to about 20000 FIP units, from about 8000 FIP units to about 15000 FIP units, or from about 10000 FIP units to about 12000 FIP units.
55. The use according to any of paras 43 or 52-54, or the gastro resistant capsule for use according to any of paras 44-54, wherein the one or more proteases are administered in a total daily dose of about 11340 FIP units.
56. The use according to any of paras 43 or 52-55, or the gastro resistant capsule for use according to any of paras 44-55, wherein bromelain is administered in a daily dose of from about 100 mg to about 1000 mg, from about 200 mg to about 900 mg, from about 300 mg to about 800 mg, from about 350 mg to about 700 mg, from about 400 mg to about 600 mg, or from about 450 mg to about 500 mg.
57. The use according to any of paras 43 or 52-56, or the gastro resistant capsule for use according to any of paras 44-56, wherein bromelain is administered in a daily dose of about 450 mg.
58. The use according to any of paras 43 or 52-57, or the gastro resistant capsule for use according to any of paras 44-57, wherein bromelain is administered in a daily dose of from about 500 FIP units to about 10000 FIP units, from about 1000 FIP units to about 8000 FIP units, from about 1500 FIP units to about 6000 FIP units, from about 2000 FIP units to about 4000 FIP units, or from about 2500 FIP units to about 3000 FIP units.
59. The use according to any of paras 43 or 52-58, or the gastro resistant capsule for use according to any of paras 44-58, wherein bromelain is administered in a daily dose of about 2700 FIP units.
60. The use according to any of paras 43 or 52-59, or the gastro resistant capsule for use according to any of paras 44-59, wherein trypsin is administered in a daily dose of from about 50 mg to about 500 mg, from about 100 mg to about 400 mg, from about 150 mg to about 350 mg, from about 175 mg to about 300 mg, or from about 200 mg to about 250 mg.
61. The use according to any of paras 43 or 52-60, or the gastro resistant capsule for use according to any of paras 44-60, wherein trypsin is administered in a daily dose of about 216 mg.
62. The use according to any of paras 43 or 52-61, or the gastro resistant capsule for use according to any of paras 44-61, wherein trypsin is administered in a daily dose of from about 2000 FIP units to about 20000 FIP units, from about 3000 FIP units to about 15000 FIP units, from about 4000 FIP units to about 12000 FIP units, from about 5000 FIP units to about 11000 FIP units, from about 6000 FIP units to about 10000 FIP units, or from about 7000 FIP units to about 9000 FIP units.
63. The use according to any of paras 43 or 52-62, or the gastro resistant capsule for use according to any of paras 44-62, wherein trypsin is administered in a daily dose of about 8640 FIP units.
64. The use according to any of paras 43 or 52-63, or the gastro resistant capsule for use according to any of paras 44-63, wherein rutin is administered in a total daily dose of from about 100 mg to about 1200 mg, from about 200 mg to about 1000 mg, from about 300 mg to about 900 mg, from about 400 mg to about 800 mg, from about 500 mg to about 700 mg.
65. The use according to any of paras 43 or 52-64, or the gastro resistant capsule for use according to any of paras 44-64, wherein rutin is administered in a total daily dose of about 600 mg.
66. The use according to any of paras 43 or 52-65, or the gastro resistant capsule for use according to any of paras 44-65, wherein the gastro resistant capsule is formulated to comprise about ½ a daily dose, about ⅓ of a daily dose, about ¼ of a daily dose, or about ⅕ of a daily dose, preferably wherein the gastro resistant capsule is formulated to comprise about ½ a daily dose, or about ⅓ of a daily dose.
67. The use according to any of paras 43 or 52-66, or the gastro resistant capsule for use according to any of paras 44-66, wherein the gastro resistant capsule is formulated to comprise about ⅓ of a daily dose.
68. The use according to any of paras 43 or 52-67, or the gastro resistant capsule for use according to any of paras 44-67, wherein five or fewer, four or fewer, or three or fewer gastro resistant capsules are administered daily.
69. The use according to any of paras 43 or 52-68, or the gastro resistant capsule for use according to any of paras 44-68, wherein two or three gastro resistant capsules are administered daily, preferably wherein three gastro resistant capsules are administered daily.
70. The use according to any of paras 43 or 52-69, or the gastro resistant capsule for use according to any of paras 44-69, wherein the resistant capsules are administered simultaneously or separately, or any combination thereof
71. The use according to any of paras 43 or 52-70, or the gastro resistant capsule for use according to any of paras 44-70, wherein the gastro resistant capsule is administered in a fasting state, preferably at least about 45 minutes before a meal.
72. The use according to any of paras 43 or 52-71, or the gastro resistant capsule for use according to any of paras 44-71, wherein the gastro resistant capsule is administered for at least 15 days.
73. Use of a gastro resistant capsule shell to deliver one or more proteases, wherein the capsule shell comprises:
(i) a water-soluble film forming polymer, in an amount of from about 65 wt % to about 97 wt %; and
(ii) a water-soluble enteric polymer, in an amount of from about 3 wt % to about 30 wt %.
74. Use of a gastro resistant capsule to deliver one or more proteases, wherein the capsule shell comprises:
   (i) a water-soluble film forming polymer, in an amount of from about 65 wt % to about 97 wt %; and
   (ii) a water-soluble enteric polymer, in an amount of from about 3 wt % to about 30 wt %
75. The use according to para 73 or 74, wherein the one or more proteases are delivered to the small intestine.
76. The use according to any of paras 73 to 75, wherein the capsule shell is defined according to any of paras 2-12.
77. The use according to any of paras 73 to 76, wherein the one or more proteases are defined according to any of paras 13-15.

1. A gastro resistant capsule comprising one or more proteases, wherein the capsule shell comprises:
(i) hydroxypropyl methylcellulose (HPMC), in an amount of from about 65 wt % to about 97 wt %; and
(ii) pectin, in an amount of from about 3 wt % to about 30 wt %.

2. The gastro resistant capsule according to claim 1, wherein the pectin has:
(i) a degree of esterification of from about 15% to about 40%;
(ii) a degree of amidation of from about 0% to about 25%; and/or
(iii) a molecular weight of from about 20 kDa to about 1000 kDa, from about 40 kDa to about 400 kDa, or from about 50 kDa to about 200 kDa.

3. The gastro resistant capsule according to claim 1, wherein the one or more proteases are selected from the group consisting of: bromelain, trypsin, papain, pancreatin, chymotrypsin, serratiopeptidase, ficain, nattokinase, and any combination thereof.

4. The gastro resistant capsule according to claim 1, wherein the gastro resistant capsule comprises the one or more proteases in a total amount of: from about 50 mg to about 1000 mg.

5. The gastro resistant capsule according to claim 1, wherein the gastro resistant capsule comprises one or more flavonoid.

6. The gastro resistant capsule according to claim 1, wherein the gastro resistant capsule comprises one or more flavonoid in a total amount of from about 50 mg to about 1000 mg.

7. The gastro resistant capsule according to claim 1, wherein the gastro resistant capsule further comprises one or more bulking agent.

8. The gastro resistant capsule according to claim 1, wherein:
the gastro resistant capsule remains substantially intact in the gastric phase.

9. A method of manufacturing a gastro resistant capsule comprising one or more proteases, the method comprising:
(a) providing a gastro resistant capsule shell comprising:
(i) HPMC, in an amount of from about 65 wt % to about 97 wt %; and
(ii) pectin, in an amount of from about 3 wt % to about 30 wt %; and
(b) filling the gastro resistant capsule shell with a capsule composition comprising one or more proteases.

10-15. (canceled)
